(19) United States
(12) Patent Application Publication
(10) Pub. No.: US 2025/0278768 A1
(43) Pub. Date: Sep. 4, 2025

(54) SYSTEMS AND METHODS FOR GENERATING A CUSTOMIZED BADGE

(71) Applicant: KPN Innovations, LLC, Lakewood, CO (US)

(72) Inventor: Kenneth Neumann, Lakewood, CO (US)

(73) Assignee: KPN Innovations, LLC, Lakewood, CO (US)

(21) Appl. No.: 18/592,570

(22) Filed: Mar. 1, 2024

(51) Int. Cl. G06Q 30/0601

(52) U.S. Cl. CPC G06Q 30/0621; G06Q 30/018; G16H 20/60; G06Q 30/0204

(57) ABSTRACT

A system and method for generating a customized badge is disclosed. The system comprises at least a processor and a memory to receive cohort data, receive alimentary array data, generate a cohort digital badge for the alimentary item of at least alimentary array data as a function of the cohort data, receive user data wherein the user data comprises biological extraction data, update the cohort digital badge to a user digital badge as a function of the biological extraction data, and display the user digital badge.

### FIELD OF THE INVENTION

The present invention generally relates to the field of alimentary item verification. In particular, the present invention is directed to a system and method for generating a customized badge.

### BACKGROUND

In today's digitally driven world, people are increasingly seeking ways to make informed decisions about their dietary choices. However, they often face challenges in accessing accurate and comprehensive information about the nutritional content, sourcing, and health implications. Existing methods for providing such information, such as paper menus, product labels, or nutrition fact panels, may be limited in their scope or accessibility, leaving people with incomplete or outdated data. This lack of transparency and clarity can impede peoples' ability to make choices that align with their dietary preferences, health goals, or ethical considerations. Additionally, the process of certifying menu items or products as meeting certain health or sustainability standards can be labor-intensive and prone to errors or inconsistencies.

### SUMMARY OF THE DISCLOSURE

In an aspect, a system for generating a customized badge is described. The system includes a memory communicatively connected to the at least a processor, the memory containing instructions configuring the processor to receive cohort data, receive alimentary array data, generate a cohort digital badge for the alimentary item of at least alimentary array data as a function of the cohort data, receive user data, wherein the user data comprises biological extraction data, update the cohort digital badge to a user digital badge as a function of the biological extraction data, and display the updated user digital badge.

In another aspect, a method for generating a customized badge is described. The method includes generating a customized badge, the method includes receiving, by at least a processor, cohort data, the method includes receiving, by the at least a processor, alimentary array data, the method includes generating, by the at least a processor, a cohort digital badge for the alimentary item of at least alimentary array data as a function of the cohort data, the method includes receiving, by the at least a processor, user data, wherein the user data comprises biological extraction data, the method includes updating, by the at least a processor, the cohort digital badge to a user digital badge as a function of the biological extraction data, the method includes displaying, by the at least a processor, an updated user digital badge.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary system for generating a customized badge;

FIG. 2 is a block diagram of an exemplary machine-learning process;

FIG. 3 is a diagram of an exemplary embodiment of neural network;

FIG. 4 is a diagram of an exemplary embodiment of a node of a neural network;

FIG. 5A is a diagram of an exemplary embodiment of a first embodiment of a user interface;

FIG. 5B is a diagram of an exemplary embodiment of a second embodiment of a user interface;

FIG. 6 is a block diagram of an exemplary embodiment of an apparatus for scoring a nutrient;

FIG. 7 is a flow diagram illustrating an exemplary method for generating a customized badge; and

FIG. 8 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for generating a customized badge. In an embodiment, the systems and methods leverage advanced data analytics, machine learning algorithms, and user interface design principles to provide consumers with personalized and comprehensive information about menu items, food products, and dietary choices.

Aspects of the present disclosure can be used to enhance transparency and accessibility in the alimentary industry, allowing consumers to make more informed decisions about their dietary intake. Aspects of the present disclosure can also be used to facilitate adherence to dietary preferences, health goals, and ethical considerations, thereby promoting healthier eating habits and overall well-being. This is so, at least in part, because the system can enable users to access real-time updates, personalized recommendations, and interactive experiences tailored to individual needs and preferences.

Aspects of the present disclosure allow for integration into existing digital platforms and restaurant operations, enabling widespread adoption and scalability. Exemplary embodiments illustrating aspects of the present disclosure are described below in the context of several specific examples.

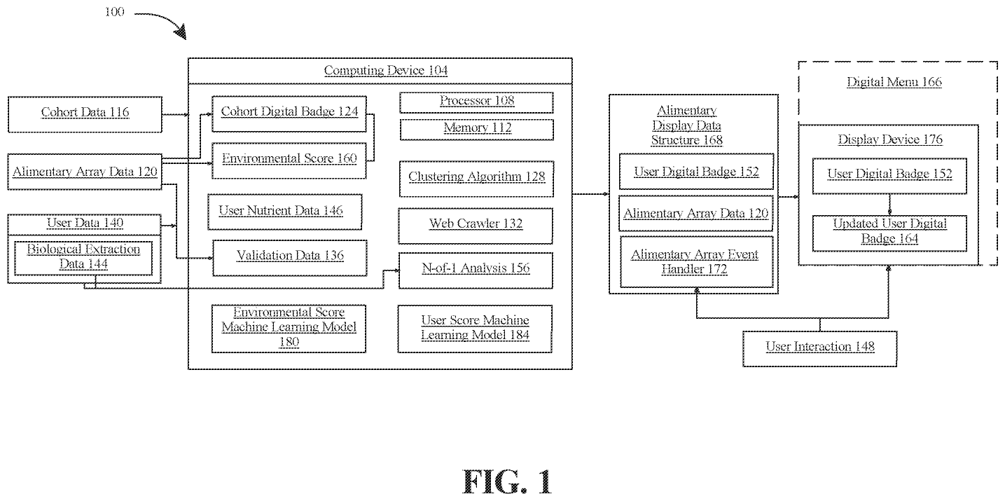

Referring now to FIG. 1, an exemplary embodiment of a system 100 for generating a customized badge is illustrated. System 100 includes a computing device 104. Computing device includes a processor 112 communicatively connected to a memory. As used in this disclosure, “communicatively connected” means connected by way of a connection, attachment or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Further referring to FIG. 1, computing device 104 may include any computing device as described in this disclosure, including without limitation a microcontroller, microprocessor, digital signal processor (DSP) and/or system on a chip (SoC) as described in this disclosure. Computing device 104 may include, be included in, and/or communicate with a mobile device such as a mobile telephone or smartphone. Computing device 104 may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented, as a non-limiting example, using a “shared nothing” architecture.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, system 100 and/or computing device may perform determinations, classification, and/or analysis steps, methods, processes, or the like as described in this disclosure using machine learning processes. A “machine learning process,” as used in this disclosure, is a process that automatedly uses a body of data known as “training data” and/or a “training set” (described further below) to generate an algorithm that will be performed by a computing device/module to produce outputs given data provided as inputs; this is in contrast to a non-machine learning software program where the commands to be executed are determined in advance by a user and written in a programming language. Machine-learning process may utilize supervised, unsupervised, lazy-learning processes and/or neural networks, described further below.

Still referring to FIG. 1, system 100 includes a memory 108 communicatively connected to at least a processor 112, the processor 112 is configured to receive cohort data 116. As used in this disclosure, “cohort data” is a collection of information that characterizes and represents a specific group of entities based on shared information. In some embodiments, badges may be specialized for user cohorts. For example, users may be grouped based on phenotype, including factors such as age, sex, nutritional needs, or chronic health conditions. System 100 may employ a classifier, which may include clustering algorithms, to classify users into distinct groups based on their shared characteristics or needs. By categorizing users into cohorts, system 100 may tailor digital badges to reflect the specific requirements or preferences of each group. As a nonlimiting example, this may include attributes, behaviors, or characteristics. The cohort data 116 may include but is not limited to demographic details, behavioral patterns, preferences, health indicators, environmental impacts, or any other measurable criteria that can be used to group entities together. In some embodiments, collecting and analyzing of cohort data 116 may identify commonalities or trends within the group that can inform decisions, predictions, or customizations of services and products, such as the generation of a customized badge (described further below). In the context of the system, in some embodiments, cohort data 116 may serve as a foundational element for tailoring digital badges to reflect the collective attributes or achievements of the group. Menu data may include nutritional information, name, and meal contents identified in a specific meal. In some embodiments, processor 112 may be configured to receive cohort data 116 by capturing comprehensive nutritional information, names, and meal contents from specific meals. In a non-limiting example, user may use system 100 to scan physical menu by employing optical character recognition (OCR). As used in this disclosure, “OCR” is a process that converts an image of text into a machine-readable text format. In some embodiments, certain alimentary provider may provide a digital menu. As used in this disclosure, “alimentary provider” is a person or entity that prepares alimentary products such as meals, food items, and/or drinks, including without limitation a restaurant, a food delivery service, or the like. In a non-limiting example, cohort data 116 may enable system 100 to automatically receive up-to-date menu data, may include detailed nutritional information and meal contents. In some embodiments, processor 112 may utilize image recognition and analysis technologies, system 100 may process photographs or scans of product labels. Captured nutritional information and ingredient lists directly from packaged food items, contributing to the menu data and received as cohort data 116.

Still referring to FIG. 1, in some embodiments, optical character recognition or optical character reader (OCR) includes automatic conversion of images of written (e.g., typed, handwritten or printed text) into machine-encoded text. In some cases, recognition of at least a keyword from an image component may include one or more processes, including without limitation optical character recognition (OCR), optical word recognition, intelligent character recognition, intelligent word recognition, and the like. In some cases, OCR may recognize written text, one glyph or character at a time. In some cases, optical word recognition may recognize written text, one word at a time, for example, for languages that use a space as a word divider. In some cases, intelligent character recognition (ICR) may recognize written text one glyph or character at a time, for instance by employing machine learning processes. In some cases, intelligent word recognition (IWR) may recognize written text, one word at a time, for instance by employing machine learning processes.

Still referring to FIG. 1, in some cases OCR may be an “offline” process, which analyses a static document or image frame. In some cases, handwriting movement analysis can be used as input to handwriting recognition. For example, instead of merely using shapes of glyphs and words, this technique may capture motions, such as the order in which segments are drawn, the direction, and the pattern of putting the pen down and lifting it. This additional information can make handwriting recognition more accurate. In some cases, this technology may be referred to as “online” character recognition, dynamic character recognition, real-time character recognition, and intelligent character recognition.

Still referring to FIG. 1, in some cases, OCR processes may employ pre-processing of image component. Pre-processing process may include without limitation de-skew, de-speckle, binarization, line removal, layout analysis or “zoning,” line and word detection, script recognition, character isolation or “segmentation,” and normalization. In some cases, a de-skew process may include applying a transform (e.g., homography or affine transform) to image component to align text. In some cases, a de-speckle process may include removing positive and negative spots and/or smoothing edges. In some cases, a binarization process may include converting an image from color or greyscale to black-and-white (i.e., a binary image). Binarization may be performed as a simple way of separating text (or any other desired image component) from a background of image component. In some cases, binarization may be required for example if an employed OCR algorithm only works on binary images. In some cases, a line removal process may include removal of non-glyph or non-character imagery (e.g., boxes and lines). In some cases, a layout analysis or “zoning” process may identify columns, paragraphs, captions, and the like as distinct blocks. In some cases, a line and word detection process may establish a baseline for word and character shapes and separate words, if necessary. In some cases, a script recognition process may, for example in multilingual documents, identify script allowing an appropriate OCR algorithm to be selected. In some cases, a character isolation or “segmentation” process may separate signal characters, for example character-based OCR algorithms. In some cases, a normalization process may normalize aspect ratio and/or scale of image component.

Still referring to FIG. 1, in some embodiments an OCR process will include an OCR algorithm. Exemplary OCR algorithms include matrix matching process and/or feature extraction processes. Matrix matching may involve comparing an image to a stored glyph on a pixel-by-pixel basis. In some case, matrix matching may also be known as “pattern matching,” “pattern recognition,” and/or “image correlation.” Matrix matching may rely on an input glyph being correctly isolated from the rest of the image component. Matrix matching may also rely on a stored glyph being in a similar font and at a same scale as input glyph. Matrix matching may work best with typewritten text.

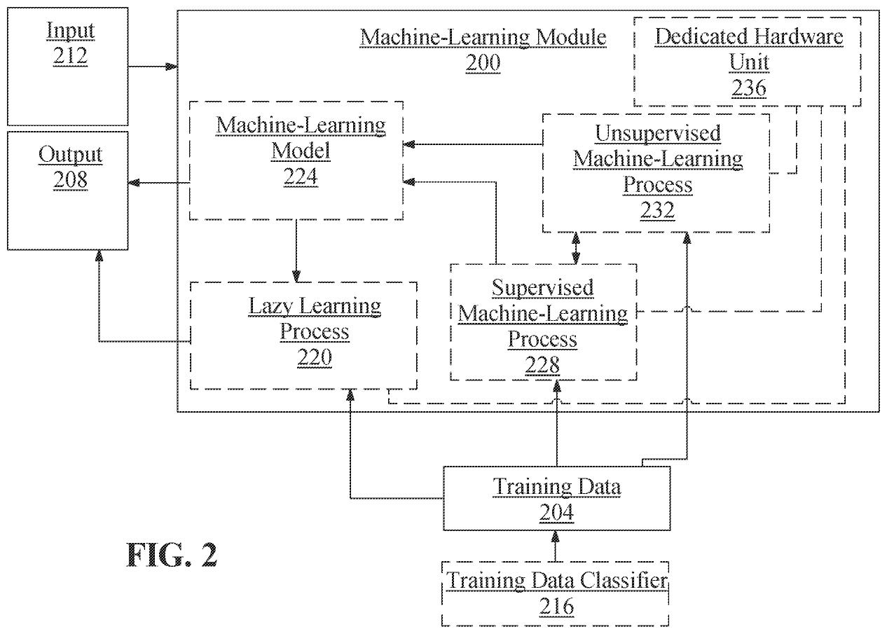

Still referring to FIG. 1, in some embodiments, an OCR process may include a feature extraction process. In some cases, feature extraction may decompose a glyph into features. Exemplary non-limiting features may include corners, edges, lines, closed loops, line direction, line intersections, and the like. In some cases, feature extraction may reduce dimensionality of representation and may make the recognition process computationally more efficient. In some cases, extracted feature can be compared with an abstract vector-like representation of a character, which might reduce to one or more glyph prototypes. General techniques of feature detection in computer vision are applicable to this type of OCR. In some embodiments, machine-learning process like nearest neighbor classifiers (e.g., k-nearest neighbors algorithm) can be used to compare image features with stored glyph features and choose a nearest match. OCR may employ any machine-learning process described in this disclosure, for example machine-learning processes described with reference to FIGS. 2-4. Exemplary non-limiting OCR software includes Cuneiform and Tesseract. Cuneiform is a multi-language, open-source optical character recognition system originally developed by Cognitive Technologies of Moscow, Russia. Tesseract is free OCR software originally developed by Hewlett-Packard of Palo Alto, California, United States.

Still referring to FIG. 1, in some cases, OCR may employ a two-pass approach to character recognition. Second pass may include adaptive recognition and use letter shapes recognized with high confidence on a first pass to recognize better remaining letters on the second pass. In some cases, two-pass approach may be advantageous for unusual fonts or low-quality image components where visual verbal content may be distorted. Another exemplary OCR software tool include OCRopus. OCRopus development is led by German Research Centre for Artificial Intelligence in Kaiserslautern, Germany. In some cases, OCR software may employ neural networks, for example neural networks as taught in reference to FIGS. 2-4.

Still referring to FIG. 1, in some cases, OCR may include post-processing. For example, OCR accuracy can be increased, in some cases, if output is constrained by a lexicon. A lexicon may include a list or set of words that are allowed to occur in a document. In some cases, a lexicon may include, for instance, all the words in the English language, or a more technical lexicon for a specific field. In some cases, an output stream may be a plain text stream or file of characters. In some cases, an OCR process may preserve an original layout of visual verbal content. In some cases, near-neighbor analysis can make use of co-occurrence frequencies to correct errors, by noting that certain words are often seen together. For example, “Washington, D.C.” is generally far more common in English than “Washington DOC.” In some cases, an OCR process may make us of a priori knowledge of grammar for a language being recognized. For example, grammar rules may be used to help determine if a word is likely to be a verb or a noun. Distance conceptualization may be employed for recognition and classification. For example, a Levenshtein distance algorithm may be used in OCR post-processing to further optimize results.

With continued reference to FIG. 1, in some embodiments, system 100 may receive cohort data 116 as the function of recipes and nutrition fact panels available online or submitted by users. In a non-limiting example, natural language processing (NLP) technique may be used to enable system 100 to parse and extract relevant data from textual recipes and standardized nutrition labels, cohort data 116 may also be translated into structured data points for meal content and nutritional profiles. In some embodiments, system 100 may facilitate a digital platform for users to manually input or submit menu data, including personal insights or unofficial nutritional information.

Still referring to FIG. 1, system 100 for generating a customized badge further comprises at least a processor 112 configured to receive alimentary array data 120. As used in this disclosure, “alimentary array data” is a collection of data related to an items, ingredients, consumable, non-consumable and/or any products provided by an alimentary provider. In a non-limiting example, items may include, but are not limited to, food, beverages, and/or any ingredient consumed at any eating occasion by an human being and/or animal. For instance and without limitation, the badge concept extends to a wide variety of products such as cosmetics (e.g., makeup, skincare products, hair care products), prescription drugs (e.g., medications prescribed by healthcare professionals for various conditions), supplements (e.g., dietary supplements, vitamins, minerals, herbal products), and electronic devices (e.g., smartphones, laptops, tablets, smartwatches, home appliances), among others. Alimentary array data 120 may encompass various attributes of alimentary items, including nutritional information, source of ingredients, preparation methods, and any certifications or validations regarding health, sustainability, or quality standards. In a non-limiting example, alimentary array data 120 may also incorporate sensory attributes of alimentary items such as taste profiles, texture descriptions, and aroma notes. These sensory attributes may provide insights into the palatability and consumer preference aspects of food items, enabling system 100 to customize digital badges not just based on nutritional or health considerations but also considering the culinary experience. Additionally, alimentary array data 120 may include consumer feedback and ratings.

Still referring to FIG. 1, processor 112 is configured to generate a cohort digital badge 124 for alimentary array data 120 of at least alimentary array data as a function of the cohort data. As described in this disclosure, a “badge” refers to an indicator denoting the quality of ingredients or components of at least an item. Initially, the badge may serve as a general certification of the item's quality based on predefined criteria. For example, in cosmetics, the badge may apply to makeup products such as foundation, lipstick, eyeshadow, and mascara, as well as skincare items such as moisturizers, cleansers, serums, and sunscreen. Hair care products such as shampoos, conditioners, styling gels, and hair oils could also feature the badge. In a further embodiment, data may be collected on an individual user, and the badge may evolve embodiments through a digital interface, incorporating personalized scoring mechanisms. Furthermore, the badge may be relevant to prescription drugs, including medications prescribed for various medical conditions such as hypertension, diabetes, asthma, and depression. Supplements like multivitamins, omega-3 fatty acids, probiotics, and herbal extracts could also be subject to the badge concept. In an additional embodiment, the badge may identify and communicate what is and isn't beneficial for the user's dietary requirements and preferences. The badge thus transforms from a generic quality indicator to a personalized guide tailored to the user's unique nutritional needs and goals. In some embodiments, the badge may include but not limited to cosmetics, prescription drugs, supplements, electronic device, and more. The badge may serve as a visual indicator certifying the quality of the product's ingredients or components based on predefined criteria. Over time, the badge may be personalized based on needs customized to a phenotype and/or N of 1 application. As used in this disclosure, a “cohort digital badge” is a visual representation indicating the alimentary item is certified for a specific group of individuals, referred to as the “cohort.” The cohort may consist of individuals sharing common characteristics, such as age group, dietary preferences, or health conditions. For instance, badge may indicate that alimentary item may be certified for individuals within the same age group as the user. By associating the badge with the cohort, users may quickly identify which alimentary items align with specific needs or preferences common to their cohort. In some embodiments, cohort digital badge 124 may be a digital symbol or recognition awarded to a defined group or category of entities (the cohort) that have achieved or demonstrated a particular standard, accomplishment, or characteristic identified through data analysis or assessment. To generate cohort digital badge, processor 112 may compare the nutritional information of alimentary item to the nutritional requirements of cohort. Nutritional requirements of cohort may also be referred to as cohort nutrient data. As used in this disclosure, “nutritional requirements” are estimated amounts of nutrients required or suggested for consumption by members of a cohort. Nutritional requirements may be focused on certain diets such as low sodium, gluten free, diabetic meal, liquid diet, low carb, low fat, low sodium, or the like. Types of cuisine may also include foods associated with a particular dieting method such as, but not limited to, Paleo Diet, the Atkins Nutritional Approach™, and the like. Comparison may be facilitated by analyzing the nutritional data associated with alimentary array data 120, including factors such as macronutrient content, caloric value, and micronutrient composition. The nutritional requirements of cohort may be determined based on carious factors, such as age, gender, dietary preferences, and health conditions. In a non-limiting example, processor 112 may utilize machine learning algorithms to analyze the nutritional information of at least one alimentary item in relation to the specific nutritional needs of cohort. Machine learning algorithms may involve statistical modeling techniques to assess the adequacy of the at least one alimentary item in meeting the dietary requirements of the target group. For example, if a dish contains an appropriate balance of macronutrients and micronutrients within the specified ranges for the cohort, cohort digital badge 124 may be generated to indicate compliance with the nutritional standards.

With continued reference to FIG. 1, in some embodiments, cohort digital badge 124 may be adapted across various fields and applications, may extend beyond alimentary providers. In some embodiments, cohort digital badge 124 may apply to educational achievements (e.g., completing a course or program), professional certifications (e.g., skills or competency levels), community engagement (e.g., participation in community service), or any other context where entities are grouped based on shared criteria or accomplishments. In a non-limiting example, processor 112 may enable software algorithms to interpret complex datasets associated with alimentary array data 120, such as nutritional information, source of ingredients, preparation methods, and various certifications or validations. In some embodiments, processor 112 may be configured to aggregate and organize alimentary array data 120 into a format conducive to analysis. In some embodiments, classifying the user into user cohorts may include allocating the user to the user cohort using a clustering algorithm 128. As used in this disclosure, a “clustering algorithm” is a computational data analysis used to group a set of objects into clusters. Objects within the same cluster may be more similar to each other than to those in other clusters. In some embodiments, similarity may be determined based on certain attributes or characteristics of the objects, which may include various types of data points or matrices. In a non-limiting example, clustering algorithm 128 may analyze user data 140 (e.g., preference, behavior, demographic information, and the like) to detect patterns and group users with similar profiles together. In some embodiments, clustering algorithm 128 may be unsupervised. In some embodiments, clustering algorithm may be supervised. In some embodiments, clustering algorithm may be trained using clustering training data; clustering training data may include sets of user data each correlated to one or more user cohorts. In some embodiments a user may be assigned to multiple cohorts. In some embodiments, user data 140/user may be manually assigned to user cohorts using a variety of rules based on data ranges. For example, for user cohorts based on age, users 20 and under may be assigned to a first user cohort, users 20 to 35 may be assigned to a second user cohort, users 35-55 may be assigned to a third user cohort, and users 55+ may be assigned to a fourth user cohort. Similar methods could be used to manually assign user cohorts based on, as non-limiting examples, weight, blood pressure, caloric intake, body fat %, and the like. In some embodiments, users may be assigned to user cohorts based on phenotype.

With continued reference to FIG. 1, in some embodiment, system 100 is further configured to use a web crawler 132 to collect validation data 136 as a function of at least alimentary array data 120. As used in this disclosure, a “web crawler” is a software program that navigates the World Wide Web in a methodical, automated manner. For example, web crawler 132 may index the content of websites across the internet by visiting specific website or online databases containing information about food items, ingredients, nutritional facts, or related data. Web crawler 132 may be programmed to extract relevant information aligning with system 100. Data may be collected from a plurality of official sources such as nutritional databases or manufacturer websites. Web crawler 132 may gather information from customer reviews and food critic reports available on various online platforms. By accessing user-generated content and professional critiques, web crawler 132 may retrieve insights into the quality, taste, and overall satisfaction of consumers regarding specific food items. In a non-limiting example, web crawler 132 may be programmed to visit specific websites, such as YELP or GOOGLE, or online databases that contain information about food items, ingredients, nutritional facts, or any other related data. In some embodiment, web crawler may search for and extract relevant information that aligns with system 100 for validation purposes. Information may include verifying the accuracy of nutritional information, sourcing methods, health certifications, or sustainability practices associated with alimentary items.

As used in this disclosure, a “validation data” is data that is used to determine whether an alimentary item meets certain requirements. Validation data 136 may take various forms depending on the context and requirements of the validation task. Validation data 136 may include factual data points, measurements, test results, certifications, expert opinions, or other forms of evidence that provide assurance or confirmation of the validity or integrity of the subject under consideration. In a non-limiting example, system 100 for generating customized badges may utilize web crawler 132 to collect validation data 136 as a function of alimentary array data 120. For instance, system 100 may aim to verify the organic certification status of alimentary items listed in its database. Web crawler 132 may be programmed to traverse relevant websites of certification agencies or regulatory bodies, systematically scanning for information related to organic certifications. Upon locating pertinent data, such as certification numbers, expiration dates, or product listings, web crawler 132 extracts and compiles validation data 136 into a structured format. Additionally, system 100 may employ data processing algorithms to cross-reference the collected validation data 136 with the corresponding alimentary array data 120, ensuring alignment and accuracy. The validation process may serve to authenticate the organic certification status of the listed alimentary items, may provide assurance to users regarding the quality and compliance of the products. In an additional embodiment, system 100 may be configured to use web crawler 132 to generate cohort badge as a function of validation data 136. For instance, user may utilize a mobile application to scan a product barcode or menu while shopping at a grocery store. The application, integrated with the system, initiates web crawling process in real-time to retrieve validation data 136 related to the scanned product or food. Web crawler 132 may navigate through reputable online sources, such as official product websites, certification databases, or consumer advocacy platforms, to gather information pertinent to the product's attributes, certifications, and quality standards.

A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Computing device 104 and/or another device may generate a classifier using a classification algorithm, defined as a process whereby a computing device 104 derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 1, computing device 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 104 may then calculate a likelihood table by calculating probabilities of different data entries and classification labels. Computing device 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, computing device 104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With continued reference to FIG. 1, validation data 136 may be classified into validation categories. A “validation category,” for the purposes of this disclosure, is a group for validation data concerning the effect of the validation data on an item's validation status. Classifying validation data 136 into a plurality of categories may involve the systematic categorization of validation information based on specific criteria or attributes. Process may be accomplished through data processing techniques and algorithms that analyze validation data 136 and assign it to predefined categories or classes. These categories may be defined based on various factors such as the type of validation, the nature of the attribute being validated, or the relevance to the system's objectives. For example, system may aim to classify validation data 136 related to food products into categories such as organic certification, fair trade certification, gluten-free certification, and non-GMO certification. As another example, validation categories may include positive feedback, neutral feedback, negative feedback, and the like. For example, a positive review left by a critic or customer may be classified to the positive feedback category. System may employ algorithms to analyze validation data 136 and identify key attributes or indicators associated with each certification type. It may then assign validation data 136 to the corresponding category based on the presence or absence of these attributes. In some embodiments, a clustering algorithm, such as any clustering algorithm described in this disclosure may be used to sort validation data 136 into validation categories. In some embodiments, clustering algorithm may be unsupervised.

With continued reference to FIG. 1, validation data 136 may be classified into validation categories using a validation classifier. In some embodiments, validation classifier may be trained using unsupervised training. In some embodiments, validation classifier may be trained using supervised training. Validation classifier may be trained using validation classification training data. Validation classification training data may include sets of validation data correlated to validation categories. In some embodiments, validation classification training data may be retrieved from a database such as a training data database.

Still referring to FIG. 1, system 100 for generating a customized badge comprises using at least a processor 112 to receive user data 140, wherein user data 140 comprises biological extraction data 144. As used in this disclosure, a “customized badge” is a visual representation tailored to an individual user based on specific attributes, preferences, achievements, or characteristics. In some embodiments, the customized badges may be dynamically generated and personalized to reflect the unique profile or status of each user. In some embodiments, customized badges may incorporate various elements such as icons, text labels, colors, and graphical symbols to communicate information about the user's accomplishments, affiliations, or traits. In additional embodiments, customized badges may serve as a means of recognition, identification, or validation within a particular context or community, providing users with a visual representation of their individuality, achievements, or affiliations. As used in this disclosure, “user data” is data describing a user or the health of a user. User data 140 may include any files, documents, images, profile pictures, messages, recordings, chat logs, transcripts, etc. As used in this disclosure, “biological extraction data” refers to any element and/or elements of data relating to or describing a biological extraction. A biological extraction may include a physically extracted sample, where a “physically extracted sample” as used in this disclosure is a sample obtained by removing and analyzing tissue and/or fluid. As used in this disclosure, “biological extraction,” may refer to an element of user data 140 corresponding to a category, including without limitation, microbiome analysis, blood test results, gut wall and food sensitivity analysis, toxicity report, medical history, biomarker, genetic or epigenetic indication, or any chemical, biological, or physiological markers of data of a user. Physically extracted sample may include without limitation a blood sample, a tissue sample, a buccal swab, a mucous sample, a stool sample, a hair sample, a fingernail sample, or the like. Physically extracted sample may include, as a non-limiting example, at least a blood sample. As a further non-limiting example, a biological extraction may include at least a genetic sample. At least a genetic sample may include a complete genome of a person or any portion thereof. At least a genetic sample may include a DNA sample and/or an RNA sample. At least a biological extraction may include an epigenetic sample, a proteomic sample, a tissue sample, a biopsy, and/or any other physically extracted sample. At least a biological extraction may include an endocrinal sample.

With continued reference to FIG. 1, user data 140 may be received from a monitoring device. As used in this disclosure “monitoring device” is an electronic device that is worn on the person of a user, such as without limitation close to and/or on the surface of the skin, wherein the device can detect, analyze, and transmit information concerning a body signal such as a vital sign, and/or ambient datum, wherein allowing immediate biofeedback to be sent to the user wearing the device. Monitoring device may include, without limitation, any device that further collects, stores, and analyzes data associated with user datums. Monitoring device may consist of, without limitation, near-body electronics, on-body electronics, in-body electronics, electronic textiles, smart watches, smart glasses, smart clothing, fitness trackers, body sensors, wearable cameras, head-mounted displays, body worn cameras, Bluetooth headsets, wristbands, smart garments, chest straps, sports watches, fitness monitors, and the like thereof. Monitoring device 120 may include, without limitation, earphones, earbuds, headsets, bras, suits, jackets, trousers, shirts, pants, socks, bracelets, necklaces, brooches, rings, jewelry, AR HMDs, VR HMDs, exoskeletons, location trackers, and gesture control wearables. Monitoring device may include blood glucose sensors. Monitoring device may include devices such as APPLE WATCH, PIXEL WATCH, FITBIT, and the like.

Still referring to FIG. 1, as a further non-limiting example, the at least a biological extraction may include a signal from at least a sensor configured to detect physiological data of a user and recording the at least a biological extraction as a function of the signal. At least a sensor may include any medical sensor and/or medical device configured to capture sensor data concerning a patient, including any scanning, radiological and/or imaging device such as without limitation x-ray equipment, computer assisted tomography (CAT) scan equipment, positron emission tomography (PET) scan equipment, any form of magnetic resonance imagery (MRI) equipment, ultrasound equipment, optical scanning equipment such as photo-plethysmographic equipment, or the like. At least a sensor may include any electromagnetic sensor, including without limitation electroencephalographic sensors, magnetoencephalographic sensors, electrocardiogramsors, electromyographic sensors, or the like. At least a sensor may include a temperature sensor. At least a sensor may include any sensor that may be included in a mobile device and/or wearable device, including without limitation a motion sensor such as an inertial measurement unit (IMU), one or more accelerometers, one or more gyroscopes, one or more magnetometers, or the like. At least a wearable and/or mobile device sensor may capture step, gait, and/or other mobility data, as well as data describing activity levels and/or physical fitness. At least a wearable and/or mobile device sensor may detect heart rate or the like. At least a sensor may detect any hematological parameter including blood oxygen level, pulse rate, heart rate, pulse rhythm, and/or blood pressure. At least a sensor may be a part of system 100 or it may be a separate device in communication with system 100.

Still referring to FIG. 1, at least a first biological extraction may include data describing one or more test results, including results of mobility tests, stress tests, dexterity tests, endocrinal tests, genetic tests, psychological tests and/or evaluations, electromyographic tests, biopsies, radiological tests, genetic tests, and/or sensory tests. A biological extraction may include biological extraction elements. As used herein, a “biological extraction element” refers to a datum within the biological extraction. In a non-limiting embodiment, some examples of biological extractions may be blood samples, teeth, hair, bone marrow, saliva, and the like. Biological extraction may be submitted by user. As used herein, “user” refers to an organism. In a non-limiting embodiment, user may submit a sample of saliva to computing device 104 so that a biological extraction analysis may be performed. Additional disclosure related to biological extractions may be found in U.S. patent application Ser. No. 16/372,512, filed on Apr. 2, 2019, and entitled “METHODS AND SYSTEMS FOR UTILIZING DIAGNOSTICS FOR INFORMED VIBRANT CONSTITUTIONAL GUIDANCE,” the entirety of which is incorporated herein by reference.

In reference to FIG. 1, computing device 104 is designed and configured to receive, from a user, at least a first element of biological extraction data. As used in this disclosure, “biological extraction,” may refer to an element of user data 140 corresponding to a category, including without limitation, microbiome analysis, blood test results, gut wall and food sensitivity analysis, toxicity report, medical history, biomarker, genetic or epigenetic indication, or any chemical, biological, or physiological markers of data of a user, including for instance, and without limitation, as described in U.S. Nonprovisional application Ser. No. 16/865,740, filed on May 4, 2020, and entitled “METHODS AND SYSTEMS FOR SYSTEM FOR NUTRITIONAL RECOMMENDATION USING ARTIFICIAL INTELLIGENCE ANALYSIS FOR IMMUNE IMPACTS, the entirety of which is incorporated herein by reference. As a non-limiting example, a plurality of user biological extraction 108 data may refer to at least any two elements of data of biological extraction 108, wherein at least an element of biological extraction 108 data corresponds to genetic data.

With continued reference to FIG. 1, in some embodiment, system may be further configured to track a user interaction 148. As used in this disclosure, a “user interaction” is engagement or activity performed by a user related to the alimentary provider or alimentary array data 120 within the system. User interaction 148 may include a range of actions, such as selecting, viewing, or reviewing specific alimentary items provided by the alimentary provider, inputting dietary preferences or restrictions, responding to recommendations, participating in surveys or feedback mechanisms about the alimentary items, or any other form of engagement that involves direct interaction between the user and the digital platform's features or content concerning the alimentary provider. In some embodiment, system 100 may be further configured to analyze user interaction 148 with alimentary array data 120. For example, a user may access a mobile application or website associated with system 100 and begins browsing through a list of alimentary items offered by various alimentary providers. As the user navigates through the platform, clicks on specific items, adds items to their cart, views product details, or applies filters to refine their search, each of these actions is tracked and recorded by system. For instance, system 100 may identify which types of alimentary items are most frequently viewed or purchased by users, which features or functionalities attract higher engagement rates, or how user behavior may vary across different demographic segments.

Still referring to FIG. 1, system 100 for generating customized badge comprises at least a processor 112 to update cohort digital badge 124 to user digital badge 152 as a function of biological extraction data 144; in other words, and without limitation, cohort digital badge may be labeled as a user digital badge, copied to an empty or previously generated instance of a user digital batch, and/or combined with and/or added to an existing user digital badge. As used in this disclosure, a “user digital badge” is a digital representation or symbol generated for an individual user, personalized based on the analysis of user data. In some embodiments, the user digital badge 152 may serve as a unique identifier or credential that reflects the user's specific health-related characteristics, achievements, or statuses derived from their biological data. The process of updating a cohort digital badge 124 to user digital badge 152 may involve tailoring the recognition or certification initially applied to a group (cohort) based on general criteria, to one that is specifically customized for an individual. The customization of digital badge may use biological extraction data 144, which may include information derived from genetic analysis, biomarker assessments, physiological measurements, or any other biological data points relevant to the user's health and well-being. In a non-limiting example, a user may regularly track their physical activity, dietary habits, and health metrics using a wearable fitness device and a mobile health app. System 100 may be equipped with algorithms and analysis tools, monitors and analyzes user biological extraction data 144 derived from these sources. In some embodiments, biological extraction data 144 may include information such as heart rate variability, blood glucose levels, sleep patterns, and genetic predispositions to certain health conditions. As user continues to engage with system 100 and accumulate more biological extraction data 144 over time, system 100 may dynamically update their cohort digital badge 124 to reflect their evolving health profile and achievements. For instance, user may consistently maintain a healthy weight, may achieve fitness milestones, and demonstrate favorable biomarker trends indicative of good cardiovascular health, system 100 may adjust cohort digital badge 124 to recognize certain accomplishments. In some embodiments, system 100 may utilize machine learning algorithms to analyze user biological extraction data 144 in relation to established health benchmarks, personalized health goals, and demographic factors. Based on analysis, system 100 may customize user's digital badge to provide personalized recommendations, insights, or certifications tailored to their unique health needs and objectives. For example, user may have a genetic predisposition to gluten sensitivity, system 100 may recommend gluten-free food options and certify them with user digital badge 152 indicating suitability for individuals with gluten intolerance. Users may also use the system to scan the menu to have a quick view of products.

With continued reference to FIG. 1, in some embodiments, generating user digital badge 152 may include performing an N-of-1 analysis 156 to user data 140 compared to cohort data 116. As used in this disclosure, “N-of-1 analysis” is an analysis that is tailored to a particular user using user-specific data. In another embodiment, system 100 may be further configured to integrate environmental data into user digital badge 152. In some embodiments, integrating the environmental data into the user digital badge may include training an environmental score machine-learning model 180 using environmental score training data, wherein the environmental score training data comprises a plurality of environmental data correlated to environmental score 160. In some embodiments, training of the environmental score machine-learning model may involve exposing it to labeled environmental data, where each data point may be associated with a known environmental score 160. During the training process, environmental score machine-learning model 180 may learns to recognize patterns and relationships within environmental data that contribute to higher or lower environmental score 160. Environmental score machine-learning model 180 may be able to infer the environmental impact of different food items based on their ingredients, sourcing methods, production processes, and other relevant factors. Environmental score machine-learning model 180 may be trained to deployed within system 100 to generate environmental score 160 for alimentary array data 120. Environmental scores 160 may be incorporated into user digital badge 152, providing users with information about the environmental sustainability of the food items they consume. As used in this disclosure, an “environmental score” is a quantitative or qualitative measure that assesses the environmental impact or sustainability performance of alimentary array data 120, such as food products or ingredients, based on their manufacturing processes, sourcing practices, and other relevant factors. The environmental score 160 may be derived from an analysis of various environmental indicators, such as carbon emissions, water usage, land use, energy consumption, waste generation, and chemical usage, associated with the production lifecycle of alimentary items. After evaluating these factors, system 100 may quantify or rate the overall sustainability performance of alimentary array data 120, assigning alimentary array data 120 a score that reflects environmental impact relative to established benchmarks or standards. For example, a food product with a lower environmental score 160 may be considered more sustainable or environmentally friendly, indicating that its production processes have minimal negative impacts on the ecosystem and natural resources. Conversely, a product with a higher environmental score 160 may signify greater environmental harm or resource depletion associated with its production methods. In another embodiment, system 100 may include incorporating environmental score 160 into cohort digital badge 124. In some embodiments, incorporating environmental score 160 into cohort digital badge 124 may enhance the badge's utility by providing users with information about the collective environmental impact of alimentary items associated with a specific group or category. This integration of environmental score 160s into the cohort digital badge 124 allows users to gain insights into the overall sustainability performance of alimentary array data 120 within the cohort, facilitating informed decision-making and promoting environmentally conscious consumption behaviors. For example, cohort digital badge 124 may be generated for a group of organic food products sourced from local farms, or it may be generated for a user as he or she scans restaurant menu. In addition to displaying traditional criteria such as nutritional value and health benefits, cohort digital badge 124 may also incorporate environmental score 160 that reflects the sustainability practices employed by the participating alimentary providers. Environmental score 160 may be calculated based on factors such as organic farming practices, use of renewable energy sources, reduction of greenhouse gas emissions, and conservation of natural resources.

With continued reference to FIG. 1, in some embodiments, integrating environmental data into user digital badge 152 may include receiving environmental data for the alimentary item. This environmental data may include information related to the environmental impact of the food item, such as its carbon footprint, water usage, energy consumption during production, sourcing methods, packaging materials, and any relevant certifications or eco-labels. By receiving and incorporating environmental data into user digital badge 152, users may gain insights into the environmental sustainability of the food items they consume, may allow users to make informed choices that align with user values and preferences. In some embodiments, environmental data may be collected using a data crawler, such as an environmental data crawler. Data crawler may be trained to crawl websites such as blogs, newspaper articles, governmental body websites, industry standard websites, industry watchdog websites, and the like. In some embodiments, environmental data may be processed using language processing algorithms. In some embodiments, this may include the use of a language processing module. Language processing module may include any hardware and/or software module. Language processing module may be configured to extract, from the one or more documents, one or more words. One or more words may include, without limitation, strings of one or more characters, including without limitation any sequence or sequences of letters, numbers, punctuation, diacritic marks, engineering symbols, geometric dimensioning and tolerancing (GD&T) symbols, chemical symbols and formulas, spaces, whitespace, and other symbols, including any symbols usable as textual data as described above. Textual data may be parsed into tokens, which may include a simple word (sequence of letters separated by whitespace) or more generally a sequence of characters as described previously. The term “token,” as used herein, refers to any smaller, individual groupings of text from a larger source of text; tokens may be broken up by word, pair of words, sentence, or other delimitation. These tokens may in turn be parsed in various ways. Textual data may be parsed into words or sequences of words, which may be considered words as well. Textual data may be parsed into “n-grams”, where all sequences of n consecutive characters are considered. Any or all possible sequences of tokens or words may be stored as “chains”, for example for use as a Markov chain or Hidden Markov Model.

Still referring to FIG. 1, language processing module may operate to produce a language processing model. Language processing model may include a program automatically generated by computing device and/or language processing module to produce associations between one or more words extracted from at least a document and detect associations, including without limitation mathematical associations, between such words. Associations between language elements, where language elements include for purposes herein extracted words, relationships of such categories to other such term may include, without limitation, mathematical associations, including without limitation statistical correlations between any language element and any other language element and/or language elements. Statistical correlations and/or mathematical associations may include probabilistic formulas or relationships indicating, for instance, a likelihood that a given extracted word indicates a given category of semantic meaning. As a further example, statistical correlations and/or mathematical associations may include probabilistic formulas or relationships indicating a positive and/or negative association between at least an extracted word and/or a given semantic meaning; positive or negative indication may include an indication that a given document is or is not indicating a category semantic meaning. Whether a phrase, sentence, word, or other textual element in a document or corpus of documents constitutes a positive or negative indicator may be determined, in an embodiment, by mathematical associations between detected words, comparisons to phrases and/or words indicating positive and/or negative indicators that are stored in memory at computing device, or the like.

Still referring to 1, language processing module and/or diagnostic engine may generate the language processing model by any suitable method, including without limitation a natural language processing classification algorithm; language processing model may include a natural language process classification model that enumerates and/or derives statistical relationships between input terms and output terms. Algorithm to generate language processing model may include a stochastic gradient descent algorithm, which may include a method that iteratively optimizes an objective function, such as an objective function representing a statistical estimation of relationships between terms, including relationships between input terms and output terms, in the form of a sum of relationships to be estimated. In an alternative or additional approach, sequential tokens may be modeled as chains, serving as the observations in a Hidden Markov Model (HMM). HMMs as used herein are statistical models with inference algorithms that that may be applied to the models. In such models, a hidden state to be estimated may include an association between an extracted words, phrases, and/or other semantic units. There may be a finite number of categories to which an extracted word may pertain; an HMM inference algorithm, such as the forward-backward algorithm or the Viterbi algorithm, may be used to estimate the most likely discrete state given a word or sequence of words. Language processing module may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

Alternatively or additionally, and with continued reference to FIG. 1, language processing module may be produced using one or more large language models (LLMs)

Continuing to refer to FIG. 1, generating language processing model may include generating a vector space, which may be a collection of vectors, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each vector in an n-dimensional vector space may be represented by an n-tuple of numerical values. Each unique extracted word and/or language element as described above may be represented by a vector of the vector space. In an embodiment, each unique extracted and/or other language element may be represented by a dimension of vector space; as a non-limiting example, each element of a vector may include a number representing an enumeration of co-occurrences of the word and/or language element represented by the vector with another word and/or language element. Vectors may be normalized, scaled according to relative frequencies of appearance and/or file sizes. In an embodiment associating language elements to one another as described above may include computing a degree of vector similarity between a vector representing each language element and a vector representing another language element; vector similarity may be measured according to any norm for proximity and/or similarity of two vectors, including without limitation cosine similarity, which measures the similarity of two vectors by evaluating the cosine of the angle between the vectors, which can be computed using a dot product of the two vectors divided by the lengths of the two vectors. Degree of similarity may include any other geometric measure of distance between vectors.

Still referring to FIG. 1, language processing module may use a corpus of documents to generate associations between language elements in a language processing module, and diagnostic engine may then use such associations to analyze words extracted from one or more documents and determine that the one or more documents indicate significance of a category. In an embodiment, language module and/or computing device 104 may perform this analysis using a selected set of significant documents, such as documents identified by one or more experts as representing good information; experts may identify or enter such documents via graphical user interface, or may communicate identities of significant documents according to any other suitable method of electronic communication, or by providing such identity to other persons who may enter such identifications into computing device 104. Documents may be entered into a computing device by being uploaded by an expert or other persons using, without limitation, file transfer protocol (FTP) or other suitable methods for transmission and/or upload of documents; alternatively or additionally, where a document is identified by a citation, a uniform resource identifier (URI), uniform resource locator (URL) or other datum permitting unambiguous identification of the document, diagnostic engine may automatically obtain the document using such an identifier, for instance by submitting a request to a database or compendium of documents such as JSTOR as provided by Ithaka Harbors, Inc. of New York.

With continued reference to FIG. 1, language processing module may be configured to identify words that are relevant to the environmental impact of an alimentary item. For example, words that appear in close proximity to phrases such as “environmentally friendly,” “CO2 Neutral,” “green,” and the like may cause the alimentary item to be assigned a higher environmental score.

With continued reference to FIG. 1, in some embodiments, integrating environmental data into user digital badge 152 may include generating environmental score 160 using environmental data and environmental score machine-learning model 180. System 100 may collect environmental data for the alimentary item, which may include factors such as carbon footprint, water usage, energy consumption, sourcing methods, and packaging materials. System 100 may then employ environmental score machine-learning model 180, trained to analyze environmental data and generate environmental score 160. Environmental score machine-learning model 180 may be trained using environmental score training data. Environmental score training data may include environmental data correlated to environmental scores 160. In some embodiments, environmental score training data may include alimentary array data 120 and environmental data correlated to environmental scores 160. In some embodiments, system 100 may periodically update environmental machine-learning model 180. System 100 may calculate environmental score for the alimentary item. Environmental score may quantify the environmental impact of the item based on the factors analyzed. Environmental score may then be incorporated into user digital badge 152, providing users with a clear indication of the environmental sustainability of the alimentary item.

With continued reference to FIG. 1, in some embodiments, generating user digital badge 152 may further include selecting a preferred alimentary data as a function of user score data, wherein the preferred alimentary data may include a higher user score than an evaluated alimentary array data. As used in this disclosure, a “preferred alimentary data” refers to alimentary items or products that are prioritized or recommended to the user based on their individual preferences, health goals, or previous interactions with the system. The preferred alimentary data may be selected as a function of user score data, may encompass various metrics, assessments, or ratings assigned to alimentary items based on their alignment with the user's preferences, dietary requirements, nutritional needs, or other relevant criteria. For example, a user may be specified dietary preferences such as vegan, gluten-free, or low-sodium, and provide feedback indicating their preference for organic and locally sourced ingredients. System may utilize machine learning algorithms and personalized scoring mechanisms to evaluate and rank alimentary array data 120 according to their compatibility with user preferences and goals. In another embodiment, generating user digital badge 152 may include using a user score machine-learning model 184 to generate the user score data. User score machine-learning model 184 may be trained using score training data. Score training data may include user data 140 and alimentary items correlated to user score data.

Still referring to FIG. 1, system 100 for generating a customized badge comprises at least a processor 112 to adjust user digital badge 152, wherein adjusting user digital badge 152 includes comparing received user data 140 with previous user data. In an embodiment, adjusting user digital badge 152 may further include comparing received user data with previous user data 140. In a further embodiment, at least a processor 112 to compare user data 140 with previous user data 140 to identify changes in the user scores. In some embodiment, user digital badge 152 may include identifying changes within the user data 140. In some embodiments, memory 108 may include instructions further configuring at least a processor 112 to adjust user digital badge 152, wherein adjusting user digital badge 152 may include receiving a digital badge machine-learning model. Digital badge machine-learning model may be designed to analyze data inputs and update user digital badge 152 accordingly. Digital badge machine-learning model may learn continuously from user interactions, feedback, and changing environmental factors to optimize the information presented in user digital badge 152. By incorporating the digital badge machine-learning model, system 100 may adapt user digital badge 152 in real-time, providing users with personalized and up-to-date information according to user needs and preferences. In another embodiments, adjusting user digital badge 152 further includes training the digital badge machine-learning model using user-specific training data. Training data for digital badge machine-learning model may include digital badge training data. Digital badge training data may include alimentary array data and user data 120 correlated to cohort digital badges. Digital badge training data may include alimentary array data and user cohorts correlated to cohort digital badges. Digital badge training data may include alimentary array data and user data 120 correlated to user digital badges. User-specific training data may be tailored to the individual user, capturing user unique preferences, dietary habits, health metrics, and other relevant information. User-specific training data may be drawn from monitoring device, or user specific training data could be drawn from user inputs. For example, user specific training data may include blood glucose data from a monitoring device (or from user input) and meal data from user input. For example, this can be used to train the digital badge machine-learning model to only assign user digital badges 152 to items that beneficially impact a particular users glucose levels. By analyzing this user-specific training data, the digital badge machine-learning model may learn and adapt to the user's behavior and needs over time. Digital badge machine-learning model may be configured to identify patterns, correlations, and trends within user data 140 to make predictions for updating user digital badge 152. Through training cycles, digital badge machine-learning model may continuously refine predictions and enhances its ability to personalize user digital badge 152 based on the specific requirements of each user. In additional embodiments, adjusting user digital badge 152 further includes generating the updated user digital badge 164 using the trained digital badge machine-learning model. In a non-limiting example, a wellness app may help users make informed dietary choices based on their individual health goals and preferences. User digital badge 152 may serve as a personalized guide, recommending suitable food options and providing nutritional insights tailored to each user's specific needs. To enhance the effectiveness of user digital badge, the app may incorporate a machine-learning model dedicated to optimizing dietary recommendations. Adjusting user digital badge 152 may involve receiving digital badge machine-learning model. In some embodiments, digital badge machine-learning model may be pre-trained using biological extraction data 144 or cohort data 116, providing a foundation for personalized recommendations. However, to tailor the recommendations more closely to the individual user's preferences and health profile, processor 112 may be further configured to train digital badge machine-learning model using user-specific training data. User-specific data may include information such as the user's dietary habits, health goals, allergies, and any other relevant factors. By adjusting the machine-learning model with data specific to the individual user, system 100 may perform an N-of-1 analysis, enabling the generation of a user digital badge that aligns more closely with the user's needs and preferences.

With continued reference to FIG. 1, cohort digital badge may be determined using cohort nutrient data. As a non-limiting example, cohort nutrient data for a user cohort may be determined. Nutrient data for each alimentary item in alimentary array data 120 may be compared against cohort nutrient data. In some embodiments, alimentary items that satisfy the nutrients of cohort nutrient data may be assigned cohort digital badge. In some embodiments, alimentary items that satisfy a percentage of nutrients of cohort nutrient data may be assigned cohort digital badge. This percentage may range from 5% to 95%. This percentage may range from 15% to 80%. This percentage may include 10%. This percentage may include 20%. This percentage may include 30%. This percentage may include 40%. This percentage may include 50%. This percentage may include 60%. This percentage may include 70%. This percentage may include 80%. This percentage may include 90%.

With continued reference to FIG. 1, user digital badge may be determined using user nutrient data 146. As a non-limiting example, user nutrient data 146 for a particular user may be determined. Nutrient data for each alimentary item in alimentary array data 120 may be compared against user nutrient data 146. In some embodiments, alimentary items that satisfy the nutrients of user nutrient data 146 may be assigned cohort digital badge. In some embodiments, alimentary items that satisfy a percentage of nutrients of user nutrient data 146 may be assigned cohort digital badge. This percentage may range from 5% to 95%. This percentage may range from 15% to 80%. This percentage may include 10%. This percentage may include 20%. This percentage may include 30%. This percentage may include 40%. This percentage may include 50%. This percentage may include 60%. This percentage may include 70%. This percentage may include 80%. This percentage may include 90%.

With continued reference to FIG. 1, preferred alimentary data may include user nutrient data 146. For the purposes of this disclosure, “user nutrient data” is data regarding recommended nutrients for a user. In some embodiments, user nutrient data 146 may be determined as a function of a user cohort, such as an age group or phenotype. In some embodiments, user nutrient data 146 may be calculated using a user's biological extraction data 144. In some embodiments, preferred alimentary data may include cohort nutrient data. For the purposes of this disclosure, “cohort nutrient data” is data regarding recommended nutrients for users in a certain cohort. In some embodiments, cohort nutrient data may be determined as a function of user cohort.

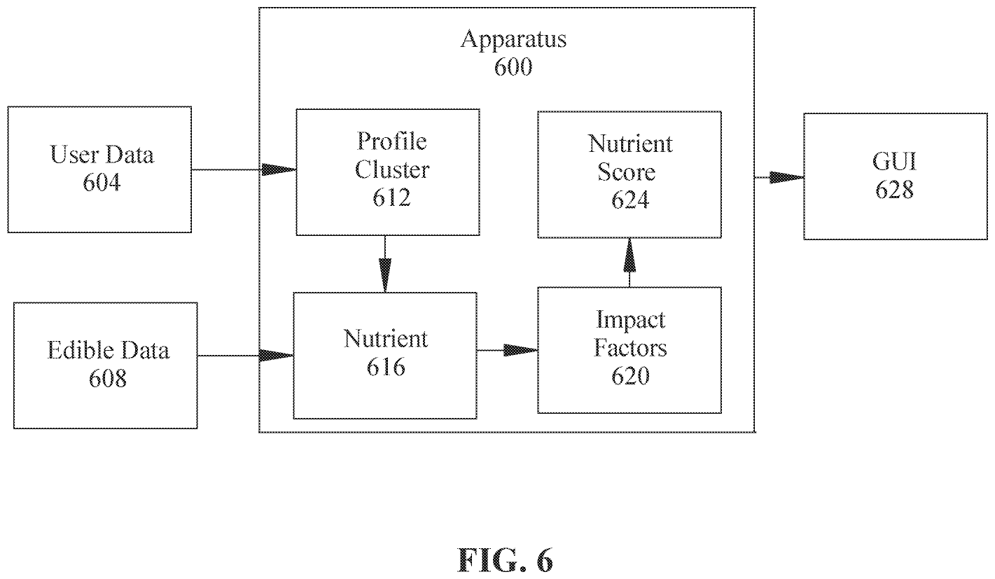

With continued reference to FIG. 1, in some embodiments, user nutrient data 146/cohort nutrient data may be determined using apparatus 600, disclosed further with reference to FIG. 6. As a non-limiting example, apparatus 600 may determine a set of nutrients for a user optimize said set of nutrients for a maximum nutrient score. Nutrient score may be a function of user data or the user cohort. In some embodiments, determination of user nutrient data 146 and/or cohort nutrient data may be conducted as disclosed in U.S. Non-provisional application Ser. No. 18/090,411 filed on Dec. 28, 2022, and entitled “APPARATUS AND METHOD FOR SCORING A NUTRIENT,” the entirety of which is incorporated by reference herein. In some embodiments, determination of user nutrient data 146 and/or cohort nutrient data may include any of the methods for recipe scoring and/or generating an ingredient chain, disclosed in U.S. Non-provisional application Ser. No. 17/976,329 filed on Oct. 28, 2022, and entitled “APPARATUS AND METHOD FOR GENERATING AN INGREDIENT CHAIN,” the entirety of which is incorporated by reference herein. In some embodiments, determination of user nutrient data 146 and/or cohort nutrient data may include any of the methods for selecting compatible elements disclosed in U.S. Non-provisional application Ser. No. 16/589,082 filed on Sep. 30, 2019, and entitled “METHODS AND SYSTEMS FOR USING ARTIFICIAL INTELLIGENCE TO SELECT A COMPATIBLE ELEMENT,” the entirety of which is incorporated by reference herein. In some embodiments, determination of user nutrient data 146 and/or cohort nutrient data may include any of the methods for identifying nutrient imbalances (particularly, based on biological extraction) and/or predicting alimentary elements disclosed in U.S. Non-provisional application Ser. No. 18/099,346 filed on Jan. 20, 2023, and entitled “METHOD AND SYSTEM FOR PREDICTING ALIMENTARY ELEMENT ORDERING BASED ON BIOLOGICAL EXTRACTION,” the entirety of which is incorporated by reference herein.

With continued reference to FIG. 1, user nutrient data may be determined using user data from a monitoring device. In some embodiments, a portion size may be determined as a function of user data from the monitoring device. As a non-limiting example, if user data indicates that a user has undertaken strenuous exercise, then a larger portion size may be recommended. In some embodiments, system 100 may determine or update user nutrient data using data from monitoring device as disclosed in U.S. Non-provisional application Ser. No. 17/833,742, filed on Jul. 6, 2022, and entitled “SYSTEM AND METHOD FOR MODIFYING A NUTRITION REQUIREMENT,” the entirety of which is incorporated herein by reference.

Still referring to FIG. 1, in an embodiment, displaying the user digital badge 152 may include generating an alimentary display data structure 168 comprising the at least alimentary array data 120 and an alimentary array event handler 172, wherein alimentary display data structure 168 may be configured to cause a display device 176 to display the alimentary array and user digital badge 152, and alimentary array event handler 172 may be configured to detect user interaction 148 on an item of the at least an alimentary array and, as a function of the detection, display a user score associated with the item. As used in this disclosure, a “display data structure” is data structure that is configured to cause a display device to display certain data. Alimentary display data structure 168 may include, without limitation, a button, a link, a checkbox, a text entry box and/or window, a drop-down list, a slider, a camera, or any other alimentary display data structure 168 that may occur to a user alimentary item upon reviewing the entirety of this disclosure. An “event handler,” as used in this disclosure, is a software function that executes an action in response to a particular event. For instance, and without limitation, an event handler may record data corresponding to user selections of previously populated fields such as drop-down lists and/or text auto-complete and/or default entries, data corresponding to user selections of checkboxes, radial buttons, or the like, potentially along with automatically entered data triggered by such selections, user entry of textual data using a keyboard, touchscreen, speech-to-text program, or the like. Event handler may generate prompts for further information, may compare data to validation rules such as requirements that the data in question be entered within certain numerical ranges, and/or may modify data and/or generate warnings to a user in response to such requirements. Event handler may convert data into expected and/or desired formats, for instance such as date formats, currency entry formats, name formats, or the like. Event handler may transmit data from display device 176 to computing device 104. As used in this disclosure, a “display device” is a hardware component responsible for presenting visual information to the user. This encompasses a wide range of electronic displays, including but not limited to monitors, screens found in laptops and smartphones, projection systems, wearable devices like smartwatches, and augmented reality glasses. Display device 176 may utilize various display technologies such as LCD, OLED, and projection systems to present images, text, and graphical elements to users. Display devices may feature touchscreens for user interaction, allowing users to navigate through digital content and interact with applications. In a non-limiting example, user may navigate through an ordering app's interface to browse different food options available from various restaurants. As the user scrolls through the list of food items, display device 176 may present alimentary display data structure 168 includes images and descriptions of the food items along with their respective user digital badges. For instance, the user may select a particular dish from a list, alimentary array event handler 172 may detect user interaction 148 and validation data 136, may then display a user score associated with the selected alimentary item. The user score may reflect the item's nutritional value, sustainability impact, or any other relevant criteria based on the user's preferences and dietary requirements. Alimentary display data structure 168 may be transmitted to display device 176. As a result, the user may view the nutritional information, user scores, and other relevant details for each alimentary item directly on their smartphone screen, helping them make informed decisions about their meal choices. In some embodiments, event handler 172 may monitor for updated user data 140. As a function of this updated user data 140, event handler may trigger the retraining of user score machine-learning model 184 and/or badge machine-learning model using the updated user data 140.

With continued reference to FIG. 1, system 100 for generating a customized badge comprises at least a processor 112 to display an updated user digital badge 164. As used in this disclosure, an “updated user digital badge” is a revised or modified version of user digital badge that incorporates the latest information, assessments, or insights relevant to the user's dietary preferences, health status, or other pertinent factors. The updated user digital badge 164 may serve as a visual representation or symbol displayed to the user, providing user with real-time feedback, guidance, or recognition related to their alimentary choices and health-related achievements. The process of displaying updated user digital badge 164 may involve presenting the revised badge on a digital platform or interface accessible to the user, such as a mobile application, website, or wearable device. Updated user digital badge 164 may allow users to conveniently view and interact with their personalized digital badge, gaining insights into their dietary habits, nutritional intake, and overall health profile. The content and user interface of updated user digital badge 164 may vary depending on the specific features and functionalities of system 100. In some embodiments, updated user digital badge 164 may include graphical elements, textual information, or interactive features that convey relevant data and insights to user in a clear and comprehensible manner. For example, updated user digital badge 164 may display nutritional information, dietary recommendations, achievement milestones, or progress towards health goals. Updated user digital badge 164 may also incorporate visual cues or color-coded indicators to highlight key aspects of the user's alimentary choices, such as adherence to dietary guidelines, consumption of specific nutrients, or alignment with personalized health objectives.

With continued reference to FIG. 1, in some embodiments, displaying the updated user digital badge 164 may include generating a digital menu as a function of the alimentary array and displaying the updated user digital badge 164 on the digital menu 166 at a display corresponding to the alimentary item. As used in this disclosure, a “digital menu” is a digital representation of a menu displayed on a screen or device, such as a smartphone, tablet, or interactive kiosk. Digital menu may include a representation of a paper menu, such as an image imitating or simulating an appearance of a digital menu. Digital menu may include information about various food or dish items available for selection, including their names, descriptions, prices, and nutritional details. Digital menu may also include images or illustrations of the items to enhance the user experience. In an embodiment, digital menu may be enhanced with the capability to display updated user digital badges alongside the alimentary items. When a user interacts with digital menu to select a specific food item, the corresponding updated user digital badge may be dynamically displayed alongside the item's information. This may allow users to quickly assess the healthiness or suitability of the selected item based on their individual dietary preferences, nutritional needs, or health goals. In a non-limiting example, the user may be dining at a restaurant equipped with the digital menu. Digital menu may provide detailed information about the available dishes and offer personalized recommendations based on users' dietary preferences and health goals. Users may decide to explore the menu and come across an option for a salad, which is part of the alimentary array presented on the digital menu. As the user taps on the salad item to view more details, digital menu may expand to reveal more information, including the updated user digital badge associated with the dish. In this example, the alimentary array refers to the collection of food items listed on the digital menu, with each item accompanied by relevant details such as ingredients, nutritional content, and user ratings. The salad is one of the alimentary items available for selection. The updated user digital badge may be displayed alongside the salad provides valuable insights into its healthfulness based on individual dietary preferences and nutritional needs. For instance, user may indicate a preference for plant-based meals and a desire to limit sodium intake, the updated user digital badge may indicate that the salad is rich in plant-based proteins and low in sodium, making it a suitable choice for the user. Furthermore, the updated user digital badge may highlight the salad's nutrient-dense ingredients, such as fresh vegetables, hearty quinoa, and a flavorful vinaigrette made with heart-healthy olive oil. This information is continuously updated in real-time, ensuring that the badge reflects the latest nutritional analysis of the dish.

Still referring to FIG. 1, in an embodiment, wherein displaying the updated user digital badge 164 may further include scanning a physical menu. As used in this disclosure, a “physical menu” is a traditional menu printed on paper or displayed in a physical format within a restaurant or dining establishment. Unlike digital menus, which are electronic and interactive, physical menus are tangible objects that patrons can hold and peruse to view the available food and beverage options offered by the establishment. To display updated user digital badge 164, scanning a physical menu may be scanned using a mobile device equipped with a scanning or imaging capability, such as a smartphone or tablet, to capture images or text from the physical menu. The scanned data may be processed by the device to identify and extract information about the menu items, including names, descriptions, and possibly nutritional details. Once the physical menu is scanned, the device may utilize image recognition or optical character recognition (OCR) technology to interpret the menu content and identify specific alimentary items listed. After identifying the items, the device may cross-reference the information with the user's dietary preferences, health goals, and any other relevant data to generate or update the user digital badge. For example, if a user scans a physical menu and selects a dish that aligns with their preferences for low-sugar and high-fiber options, the updated user digital badge displayed on user device may indicate the nutritional attributes of the selected item, such as its fiber content and absence of added sugars. This allows the user to make informed choices based on their individual dietary requirements, even when presented with a traditional physical menu.

With continued reference to FIG. 1, in an additional embodiment, displaying the updated user digital badge 164 may further include identifying a listing on the physical menu corresponding to the alimentary item. This process may ensure that the information presented on updated user digital badge 164 accurately reflects the chosen menu item, allowing users to make informed decisions about their food choices based on their individual preferences and dietary requirements. In a non-limiting example, user may select the salad option from the physical menu, user may use a mobile device equipped with scanning capabilities to capture an image of the menu listing or manually input the name of the salad into a digital interface. System 100 may then identify the corresponding listing on the physical menu, which may include details such as the salad's name, description, and price. Once the listing is identified, system 100 may cross-references this information with the user's dietary preferences and health goals to generate updated user digital badge 164 for the selected salad. Updated user digital badge 164 may display relevant nutritional information, for example, the salad's calorie count, macronutrient composition, and any special dietary attributes (e.g., gluten-free, vegan). The salad may be the listing on the physical menu corresponds to the specific alimentary item chosen by the user. By identifying the menu listing, system 100 may ensure the information displayed on updated user digital badge 164 aligns with the user's selection, enabling them to make informed decisions about their meal based on their individual dietary needs and preferences.

With continued reference to FIG. 1, in some embodiments, displaying the updated user digital badge 164 may further include generating a combined display of the physical menu and the updated user digital badge, wherein the combined display displays the updated user digital badge at the listing and displaying the combined display. The combined display may enhance the user experience by providing comprehensive information about the selected menu item directly alongside its listing on the physical menu. For example, user may be browsing the physical menu at a restaurant and select a particular dish. Upon selecting the item, the digital system may overlay updated user digital badge 164 onto the listing of the chosen item, creating a combined display. This overlay may be achieved using augmented reality (AR) technology, where updated user digital badge 164 appears as a digital layer superimposed onto the physical menu. The combined display may integrate updated user digital badge 164 with the physical menu, may allow users to view essential information about the selected item without having to navigate away from the menu itself. Updated user digital badge 164 may provide details such as the dish's nutritional content, ingredient list, allergen information, and any special dietary attributes.

Referring now to FIG. 2, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm instantiated in hardware or software logic, data structures, and/or functions that will be performed by a computing device/module to produce outputs 208 given data provided as inputs 212; this is in contrast to a non-machine learning software program where the commands to be executed are determined in advance by a user and written in a programming language.

Still referring to FIG. 2, “training data,” as used herein, is data containing correlations that a machine-learning process may use to model relationships between two or more categories of data elements. For instance, and without limitation, training data 204 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure. As a non-limiting example, illustrative inputs may consist of nutritional data obtained from menu items, such as calorie content, macronutrient composition, and presence of allergens. Outputs may then correspond to the generation of digital badges indicating the healthiness level of each menu item, ranging from “healthy” to “less healthy,” based on predefined thresholds or criteria established by the machine-learning model. Similarly, inputs related to ingredient sourcing and environmental impact may lead to outputs in the form of sustainability scores integrated into the digital badges, guiding consumers toward environmentally-friendly food choices.

Further referring to FIG. 2, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 200 may generate a classifier using a classification algorithm, defined as a process whereby a computing device and/or any module and/or component operating thereon derives a classifier from training data 204. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 216 may classify elements of training data to characterize sub-populations such as cohorts of persons or other analyzed items or phenomena. Classifications may allow for the selection of specific subsets of training data tailored to the characteristics of each cohort. Dietary preference cohorts could group users based on their dietary choices, ensuring that recommendations and badges align with individual preferences, whether vegetarian, vegan, gluten-free, or otherwise.

Still referring to FIG. 2, computing device 204 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 204 may then calculate a likelihood table by calculating probabilities of different data entries and classification labels. Computing device 204 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 2, computing device 204 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 2, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 2, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 2, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 2, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 2, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 2, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 2, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 2, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 2, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{new} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{new} = \frac{X - {X}_{mean}}{{X}_{max} - {X}_{min}} .$$

Feature scaling may include standardization, where a difference between X and Xₘₑₐₙ is divided by a standard deviation σ of a set or subset of values:

$${X}_{new} = \frac{X - {X}_{mean}}{σ} .$$

Scaling may be performed using a median value of a set or subset X_{median} and/or interquartile range (IQR), which represents the difference between the 25^{th} percentile value and the 50^{th} percentile value (or closest values thereto by a rounding protocol), such as:

$${X}_{new} = \frac{X - {X}_{median}}{IQR} .$$

Persons skinea in the art, upon reviewing the entirety of this disclosure, will be aware of various alternative or additional approaches that may be used for feature scaling.

Further referring to FIG. 2, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 2, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 2, machine-learning processes as described in this disclosure may be used to generate machine-learning models 224. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 2, machine-learning algorithms may include at least a supervised machine-learning process 228. At least a supervised machine-learning process 228, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include input as described above as inputs, output as described in this disclosure as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

With further reference to FIG. 2, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 2, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 2, machine learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 232 may not require a response variable; unsupervised processes 232 may be used to find interesting patterns and/or inferences between variables, to determine a degree of correlation between two or more variables, or the like.

Still referring to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 2, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 2, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 2, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 2, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 2, one or more processes or algorithms described above may be performed by at least a dedicated hardware unit 236. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 236 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 236 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 236 to perform one or more operations described herein, such as evaluation of model and/or algorithm outputs, one-time or iterative updates to parameters, coefficients, weights, and/or biases, and/or any other operations such as vector and/or matrix operations as described in this disclosure.

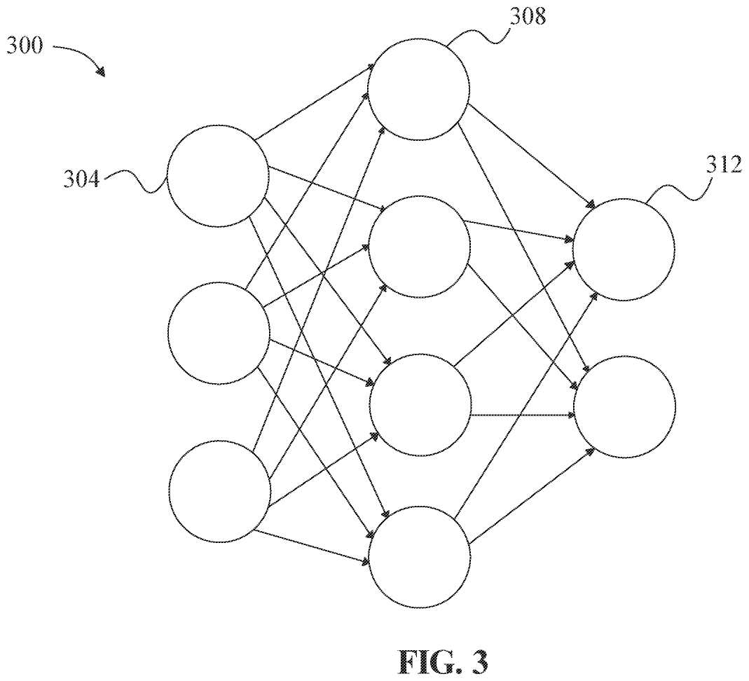

Referring now to FIG. 3, an exemplary embodiment of neural network 300 is illustrated. A neural network 300 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 304, one or more intermediate layers 308, and an output layer of nodes 312. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

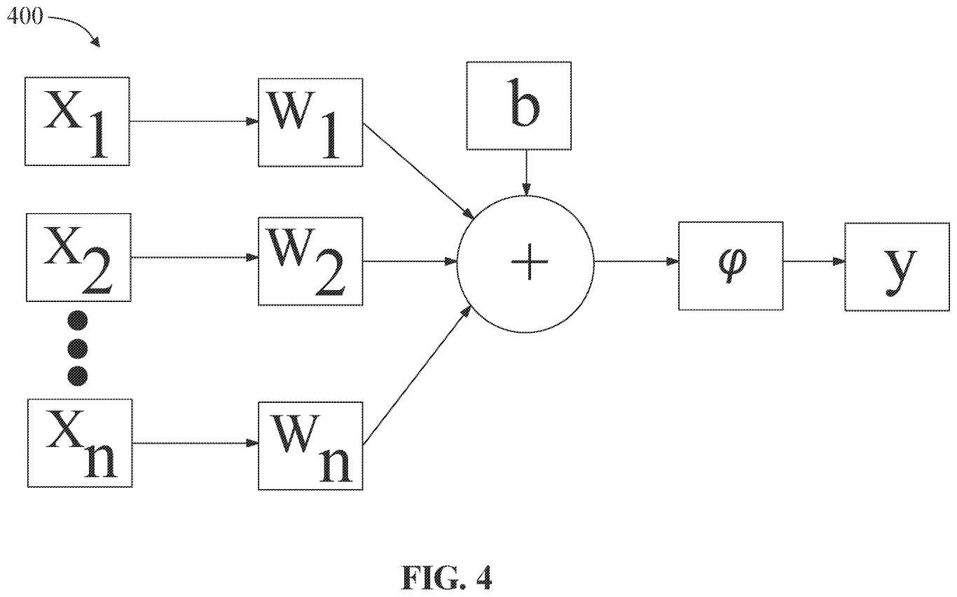

Referring now to FIG. 4, an exemplary embodiment of a node 400 of a neural network is illustrated. A node may include, without limitation a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as f(x)=tanh²(x), a rectified linear unit function such as f(x)=max(0, x), a “leaky” and/or “parametric” rectified linear unit function such as f(x)=max(ax, x) for some a, an exponential linear units function such as

$$f ( x ) = \{ \begin{matrix}x & for x \geq 0 \\ α ( {e}^{x} - 1 ) & for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as f(x)=x*sigmoid(x), a Gaussian error linear unit function such as f(x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) & for x < 0 \\ x & for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

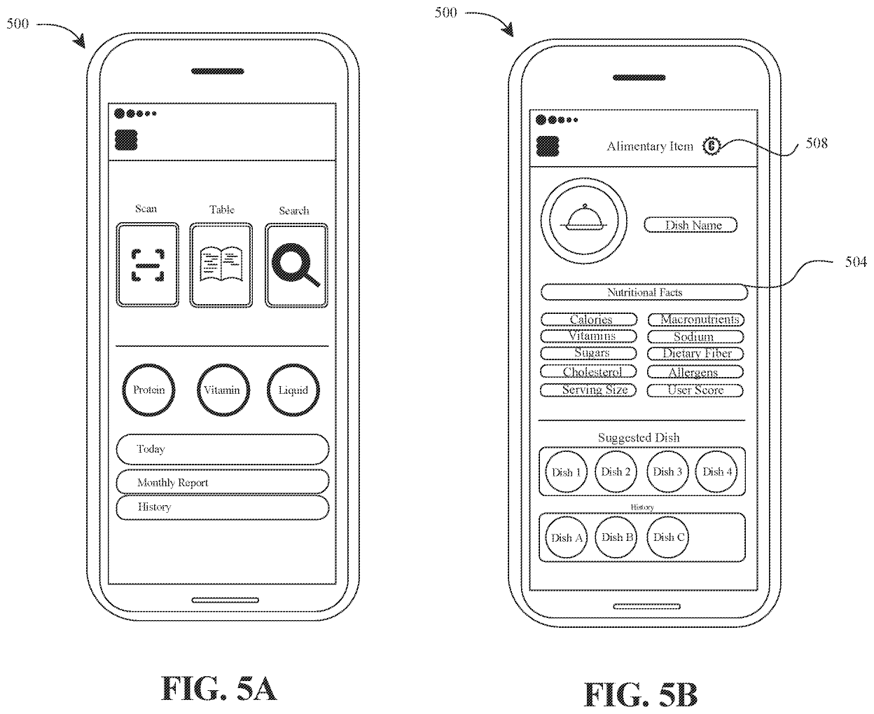

Referring now to FIG. 5A, a first embodiment of user interface 500 is shown. User interface 500 may show the user digital badge within an alimentary array, enhancing personal dietary tracking. User interface 500 may feature a lookup table that supports functionalities such as search, QR code scanning, and menu navigation. It is designed to display comprehensive dietary information, including historical and current restaurant visits, daily, weekly, or monthly consumption metrics for proteins, vitamins, and liquids, as well as integrating a detailed monthly report and consumption history. User interface setup facilitates dietary choices and aids in long-term nutritional planning and monitoring.

Referring now to FIG. 5B, a second embodiment of user interface 500 is shown. User interface 500 may display detailed information about a selected alimentary item. In some embodiments, nutrient information 504 for a selected alimentary item may be shown. This may include nutritional facts such as calories, vitamins, sugars, cholesterol, serving size, macronutrients, sodium, dietary fiber, allergens and the like. Additionally, a user score may be displayed. User score may rate the dish based on the user's health profile, suggested alternative dishes for consideration, and a history section listing previously selected dishes.

With continued reference to FIGS. 5A and 5B, user interface 500 may include a badge 508. Digital badge 508 may include a user digital badge and/or a cohort digital badge. Digital badge 508 may include any digital badge disclosed throughout this disclosure. Digital badge 508 may include an image. Image may include any suitable image such as, but not limited to, checkmarks, thumbs up, logos, ribbons, medals, and the like. Digital badge 508 may be interactive. A user may select digital badge 508 to view further nutritional information about an alimentary item or to view user score data. In some embodiments, digital badge 508 may include an event handler, as discussed above with reference to FIG. 1. In some embodiments, digital badge 508 may include a link, such as a hyperlink. In some embodiments, alimentary array data 120 may be received by system 100 in an initial alimentary array data structure. In some embodiments, digital badge 508 may be inserted into alimentary array data structure. In some embodiments, system 100 may locate the relevant alimentary item in alimentary array data structure and insert digital badge 508 into alimentary array data structure at that location. In some embodiments, this may include the use of a code injector. A code injector, for the purposes of this disclosure, is software configured to inject data into an application. Code injector may inject, as non-limiting examples. HTML, CSS, or Javascript code.

Referring now to FIG. 6, an exemplary embodiment of an apparatus 600 for scoring a nutrient is illustrated. Apparatus 600 may include a computing device as disclosed above.

With continued reference to FIG. 6, apparatus 600 may be designed and/or configured to perform any method, method step, or sequence of method steps in any embodiment described in this disclosure, in any order and with any degree of repetition. For instance, apparatus 600 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Apparatus 600 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 6, in some embodiments, apparatus 600 may be configured to receive user data 604. A “user,” as used in this disclosure, is an individual. User data 604 may include a biological extraction. A “biological extraction” as used in this disclosure includes at least an element of user physiological data. As used in this disclosure, “physiological data” is any data indicative of a person's physiological state; physiological state may be evaluated with regard to one or more measures of health of a person's body, one or more systems within a person's body such as a circulatory system, a digestive system, a nervous system, or the like, one or more organs within a person's body, and/or any other subdivision of a person's body useful for diagnostic or prognostic purposes. For instance, and without limitation, a particular set of biomarkers, test results, and/or biochemical information may be recognized in a given medical field as useful for identifying various disease conditions or prognoses within a relevant field. As a non-limiting example, and without limitation, physiological data describing red blood cells, such as red blood cell count, hemoglobin levels, hematocrit, mean corpuscular volume, mean corpuscular hemoglobin, and/or mean corpuscular hemoglobin concentration may be recognized as useful for identifying various conditions such as dehydration, high testosterone, nutrient deficiencies, kidney dysfunction, chronic inflammation, anemia, and/or blood loss. In some embodiments, user data may include physiological data.

With continued reference to FIG. 6, physiological state data may include, without limitation, hematological data, such as red blood cell count, which may include a total number of red blood cells in a person's blood and/or in a blood sample, hemoglobin levels, hematocrit representing a percentage of blood in a person and/or sample that is composed of red blood cells, mean corpuscular volume, which may be an estimate of the average red blood cell size, mean corpuscular hemoglobin, which may measure average weight of hemoglobin per red blood cell, mean corpuscular hemoglobin concentration, which may measure an average concentration of hemoglobin in red blood cells, platelet count, mean platelet volume which may measure the average size of platelets, red blood cell distribution width, which measures variation in red blood cell size, absolute neutrophils, which measures the number of neutrophil white blood cells, absolute quantities of lymphocytes such as B-cells, T-cells, Natural Killer Cells, and the like, absolute numbers of monocytes including macrophage precursors, absolute numbers of eosinophils, and/or absolute counts of basophils. Physiological state data may include, without limitation, immune function data such as Interleukine-6 (IL-6), TNF-alpha, systemic inflammatory cytokines, and the like.

Continuing to refer to FIG. 6, physiological state data may include, without limitation, data describing blood-born lipids, including total cholesterol levels, high-density lipoprotein (HDL) cholesterol levels, low-density lipoprotein (LDL) cholesterol levels, very low-density lipoprotein (VLDL) cholesterol levels, levels of triglycerides, and/or any other quantity of any blood-born lipid or lipid-containing substance. Physiological state data may include measures of glucose metabolism such as fasting glucose levels and/or hemoglobin A1-C (HbA1c) levels. Physiological state data may include, without limitation, one or more measures associated with endocrine function, such as without limitation, quantities of dehydroepiandrosterone (DHEAS), DHEA-Sulfate, quantities of cortisol, ratio of DHEAS to cortisol, quantities of testosterone quantities of estrogen, quantities of growth hormone (GH), insulin-like growth factor 1 (IGF-1), quantities of adipokines such as adiponectin, leptin, and/or ghrelin, quantities of somatostatin, progesterone, or the like. Physiological state data may include measures of estimated glomerular filtration rate (eGFR). Physiological state data may include quantities of C-reactive protein, estradiol, ferritin, folate, homocysteine, prostate-specific Ag, thyroid-stimulating hormone, vitamin D, 25 hydroxy, blood urea nitrogen, creatinine, sodium, potassium, chloride, carbon dioxide, uric acid, albumin, globulin, calcium, phosphorus, alkaline phosphatase, alanine amino transferase, aspartate amino transferase, lactate dehydrogenase (LDH), bilirubin, gamma-glutamyl transferase (GGT), iron, and/or total iron binding capacity (TIBC), or the like. Physiological state data may include antinuclear antibody levels. Physiological state data may include aluminum levels. Physiological state data may include arsenic levels. Physiological state data may include levels of fibrinogen, plasma cystatin C, and/or brain natriuretic peptide.

Continuing to refer to FIG. 6, physiological state data may include measures of lung function such as forced expiratory volume, one second (FEV-1) which measures how much air can be exhaled in one second following a deep inhalation, forced vital capacity (FVC), which measures the volume of air that may be contained in the lungs. Physiological state data may include a measurement of blood pressure, including without limitation systolic and diastolic blood pressure. Physiological state data may include a measure of waist circumference. Physiological state data may include body mass index (BMI). Physiological state data may include one or more measures of bone mass and/or density such as dual-energy x-ray absorptiometry. Physiological state data may include one or more measures of muscle mass. Physiological state data may include one or more measures of physical capability such as without limitation measures of grip strength, evaluations of standing balance, evaluations of gait speed, pegboard tests, timed up and go tests, and/or chair rising tests.

Still viewing FIG. 6, physiological state data may include one or more measures of cognitive function, including without limitation Rey auditory verbal learning test results, California verbal learning test results, NIH toolbox picture sequence memory test, Digital symbol coding evaluations, and/or Verbal fluency evaluations. Physiological state data may include one or more evaluations of sensory ability, including measures of audition, vision, olfaction, gustation, vestibular function and pain.

Continuing to refer to FIG. 6, physiological state data may include psychological data. Psychological data may include any data generated using psychological, neuro-psychological, and/or cognitive evaluations, as well as diagnostic screening tests, personality tests, personal compatibility tests, or the like; such data may include, without limitation, numerical score data entered by an evaluating professional and/or by a subject performing a self-test such as a computerized questionnaire. Psychological data may include textual, video, or image data describing testing, analysis, and/or conclusions entered by a medical professional such as without limitation a psychologist, psychiatrist, psychotherapist, social worker, a medical doctor, or the like. Psychological data may include data gathered from user interactions with persons, documents, and/or computing devices 604; for instance, user patterns of purchases, including electronic purchases, communication such as via chat-rooms or the like, any textual, image, video, and/or data produced by the subject, any textual image, video and/or other data depicting and/or describing the subject, or the like. Any psychological data and/or data used to generate psychological data may be analyzed using machine-learning and/or language processing module as described in this disclosure.

Still referring to FIG. 6, physiological state data may include genomic data, including deoxyribonucleic acid (DNA) samples and/or sequences, such as without limitation DNA sequences contained in one or more chromosomes in human cells. Genomic data may include, without limitation, ribonucleic acid (RNA) samples and/or sequences, such as samples and/or sequences of messenger RNA (mRNA) or the like taken from human cells. Genetic data may include telomere lengths. Genomic data may include epigenetic data including data describing one or more states of methylation of genetic material. Physiological state data may include proteomic data, which as used herein is data describing all proteins produced and/or modified by an organism, colony of organisms, or system of organisms, and/or a subset thereof. Physiological state data may include data concerning a microbiome of a person, which as used herein includes any data describing any microorganism and/or combination of microorganisms living on or within a person, including without limitation biomarkers, genomic data, proteomic data, and/or any other metabolic or biochemical data useful for analysis of the effect of such microorganisms on other physiological state data of a person, as described in further detail below.

With continuing reference to FIG. 6, physiological state data may include one or more user-entered descriptions of a person's physiological state. One or more user-entered descriptions may include, without limitation, user descriptions of symptoms, which may include without limitation current or past physical, psychological, perceptual, and/or neurological symptoms, user descriptions of current or past physical, emotional, and/or psychological problems and/or concerns, user descriptions of past or current treatments, including therapies, nutritional regimens, exercise regimens, pharmaceuticals or the like, or any other user-entered data that a user may provide to a medical professional when seeking treatment and/or evaluation, and/or in response to medical intake papers, questionnaires, questions from medical professionals, or the like. Physiological state data may include any physiological state data, as described above, describing any multicellular organism living in or on a person including any parasitic and/or symbiotic organisms living in or on the persons; non-limiting examples may include mites, nematodes, flatworms, or the like. Examples of physiological state data described in this disclosure are presented for illustrative purposes only and are not meant to be exhaustive.

With continued reference to FIG. 6, physiological data may include, without limitation any result of any medical test, physiological assessment, cognitive assessment, psychological assessment, or the like. Apparatus 600 may receive at least a physiological data from one or more other devices after performance; apparatus 600 may alternatively or additionally perform one or more assessments and/or tests to obtain at least a physiological data, and/or one or more portions thereof, on apparatus 600. For instance, at least physiological data may include or more entries by a user in a form or similar graphical user interface object; one or more entries may include, without limitation, user responses to questions on a psychological, behavioral, personality, or cognitive test. For instance, at least a server may present to user a set of assessment questions designed or intended to evaluate a current state of mind of the user, a current psychological state of the user, a personality trait of the user, or the like; at least a server may provide user-entered responses to such questions directly as at least a physiological data and/or may perform one or more calculations or other algorithms to derive a score or other result of an assessment as specified by one or more testing protocols, such as automated calculation of a Stanford-Binet and/or Wechsler scale for IQ testing, a personality test scoring such as a Myers-Briggs test protocol, or other assessments that may occur to persons skilled in the art upon reviewing the entirety of this disclosure.

With continued reference to FIG. 6, assessment and/or self-assessment data, and/or automated or other assessment results, obtained from a third-party device; third-party device may include, without limitation, a server or other device (not shown) that performs automated cognitive, psychological, behavioral, personality, or other assessments. Third-party device may include a device operated by an informed advisor. An informed advisor may include any medical professional who may assist and/or participate in the medical treatment of a user. An informed advisor may include a medical doctor, nurse, physician assistant, pharmacist, yoga instructor, nutritionist, spiritual healer, meditation teacher, fitness coach, health coach, life coach, and the like.

With continued reference to FIG. 6, physiological data may include data describing one or more test results, including results of mobility tests, stress tests, dexterity tests, endocrinal tests, genetic tests, and/or electromyographic tests, biopsies, radiological tests, genetic tests, and/or sensory tests. Persons skilled in the art, upon reviewing the entirety of this disclosure, will be aware of various additional examples of at least a physiological sample consistent with this disclosure.

With continued reference to FIG. 6, physiological data may include one or more user body measurements. A “user body measurement” as used in this disclosure, includes a measurable indicator of the severity, absence, and/or presence of a disease state. A “disease state” as used in this disclosure, includes any harmful deviation from the normal structural and/or function state of a human being. A disease state may include any medical condition and may be associated with specific symptoms and signs. A disease state may be classified into different types including infectious diseases, deficiency diseases, hereditary diseases, and/or physiological diseases. For instance and without limitation, internal dysfunction of the immune system may produce a variety of different diseases including immunodeficiency, hypersensitivity, allergies, and/or autoimmune disorders.

With continued reference to FIG. 6, user body measurements may be related to particular dimensions of the human body. A “dimension of the human body” as used in this disclosure, includes one or more functional body systems that are impaired by disease in a human body and/or animal body. Functional body systems may include one or more body systems recognized as attributing to root causes of disease by functional medicine practitioners and experts. A “root cause” as used in this disclosure, includes any chain of causation describing underlying reasons for a particular disease state and/or medical condition instead of focusing solely on symptomatology reversal. Root cause may include chains of causation developed by functional medicine practices that may focus on disease causation and reversal. For instance and without limitation, a medical condition such as diabetes may include a chain of causation that does not include solely impaired sugar metabolism but that also includes impaired hormone systems including insulin resistance, high cortisol, less than optimal thyroid production, and low sex hormones. Diabetes may include further chains of causation that include inflammation, poor diet, delayed food allergies, leaky gut, oxidative stress, damage to cell membranes, and dysbiosis. Dimensions of the human body may include but are not limited to epigenetics, gut-wall, microbiome, nutrients, genetics, and/or metabolism.

With continued reference to FIG. 6, epigenetic, as used herein, includes any user body measurements describing changes to a genome that do not involve corresponding changes in nucleotide sequence. Epigenetic body measurement may include data describing any heritable phenotypic. Phenotype, as used herein, may include any observable trait of a user including morphology, physical form, and structure. Phenotype may include a user's biochemical and physiological properties, behavior, and products of behavior. Behavioral phenotypes may include cognitive, personality, and behavior patterns. This may include effects on cellular and physiological phenotypic traits that may occur due to external or environmental factors. For example, DNA methylation and histone modification may alter phenotypic expression of genes without altering underlying DNA sequence. Epigenetic body measurements may include data describing one or more states of methylation of genetic material.

With continued reference to FIG. 6, gut-wall, as used herein, includes the space surrounding the lumen of the gastrointestinal tract that is composed of four layers including the mucosa, submucosa, muscular layer, and serosa. The mucosa contains the gut epithelium that is composed of goblet cells that function to secrete mucus, which aids in lubricating the passage of food throughout the digestive tract. The goblet cells also aid in protecting the intestinal wall from destruction by digestive enzymes. The mucosa includes villi or folds of the mucosa located in the small intestine that increase the surface area of the intestine. The villi contain a lacteal, that is a vessel connected to the lymph system that aids in removal of lipids and tissue fluids. Villi may contain microvilli that increase the surface area over which absorption can take place. The large intestine lack villi and instead a flat surface containing goblet cells are present.

With continued reference to FIG. 6, gut-wall includes the submucosa, which contains nerves, blood vessels, and elastic fibers containing collagen. Elastic fibers contained within the submucosa aid in stretching the gastrointestinal tract with increased capacity while also maintaining the shape of the intestine. Gut-wall includes muscular layer which contains smooth muscle that aids in peristalsis and the movement of digested material out of and along the gut. Gut-wall includes the serosa which is composed of connective tissue and coated in mucus to prevent friction damage from the intestine rubbing against other tissue. Mesenteries are also found in the serosa and suspend the intestine in the abdominal cavity to stop it from being disturbed when a person is physically active.

With continued reference to FIG. 6, gut-wall body measurement may include data describing one or more test results including results of gut-wall function, gut-wall integrity, gut-wall strength, gut-wall absorption, gut-wall permeability, intestinal absorption, gut-wall barrier function, gut-wall absorption of bacteria, gut-wall malabsorption, gut-wall gastrointestinal imbalances and the like.

With continued reference to FIG. 6, gut-wall body measurement may include any data describing blood test results of creatinine levels, lactulose levels, zonulin levels, and mannitol levels. Gut-wall body measurement may include blood test results of specific gut-wall body measurements including d-lactate, endotoxin lipopolysaccharide (LPS) Gut-wall body measurement may include data breath tests measuring lactulose, hydrogen, methane, lactose, and the like. Gut-wall body measurement may include blood test results describing blood chemistry levels of albumin, bilirubin, complete blood count, electrolytes, minerals, sodium, potassium, calcium, glucose, blood clotting factors,

With continued reference to FIG. 6, gut-wall body measurement may include one or more stool test results describing presence or absence of parasites, firmicutes, Bacteroidetes, absorption, inflammation, food sensitivities. Stool test results may describe presence, absence, and/or measurement of acetate, aerobic bacterial cultures, anerobic bacterial cultures, fecal short chain fatty acids, beta-glucuronidase, cholesterol, chymotrypsin, fecal color, Cryptosporidium EIA, Entamoeba histolytica, fecal lactoferrin, Giardia lamblia EIA, long chain fatty acids, meat fibers and vegetable fibers, mucus, occult blood, parasite identification, phospholipids, propionate, putrefactive short chain fatty acids, total fecal fat, triglycerides, yeast culture, n-butyrate, pH and the like.

With continued reference to FIG. 6, gut-wall body measurement may include one or more stool test results describing presence, absence, and/or measurement of microorganisms including bacteria, archaea, fungi, protozoa, algae, viruses, parasites, worms, and the like. Stool test results may contain species such as Bifidobacterium species, Campylobacter species, Clostridium difficile, Cryptosporidium species, Cyclospora cayetanensis, Cryptosporidium EIA, Dientamoeba fragilis, Entamoeba histolytica, Escherichia coli, Entamoeba histolytica, Giardia, H. pylori, Candida albicans, Lactobacillus species, worms, macroscopic worms, mycology, protozoa, Shiga toxin E. coli, and the like.

With continued reference to FIG. 6, gut-wall body measurement may include one or more microscopic ova exam results, microscopic parasite exam results, protozoan polymerase chain reaction test results and the like. Gut-wall body measurement may include enzyme-linked immunosorbent assay (ELISA) test results describing immunoglobulin G (Ig G) food antibody results, immunoglobulin E (Ig E) food antibody results, Ig E mold results, IgG spice and herb results. Gut-wall body measurement may include measurements of calprotectin, eosinophil protein x (EPX), stool weight, pancreatic elastase, total urine volume, blood creatinine levels, blood lactulose levels, blood mannitol levels.

With continued reference to FIG. 6, gut-wall body measurement may include one or more elements of data describing one or more procedures examining gut including for example colonoscopy, endoscopy, large and small molecule challenge and subsequent urinary recovery using large molecules such as lactulose, polyethylene glycol-3350, and small molecules such as mannitol, L-rhamnose, polyethyleneglycol-300. Gut-wall body measurement may include data describing one or more images such as x-ray, MRI, CT scan, ultrasound, standard barium follow-through examination, barium enema, barium with contract, MRI fluoroscopy, positron emission tomography 9PET), diffusion-weighted MRI imaging, and the like.

With continued reference to FIG. 6, microbiome, as used herein, includes ecological community of commensal, symbiotic, and pathogenic microorganisms that reside on or within any of a number of human tissues and biofluids. For example, human tissues and biofluids may include the skin, mammary glands, placenta, seminal fluid, uterus, vagina, ovarian follicles, lung, saliva, oral mucosa, conjunctiva, biliary, and gastrointestinal tracts. Microbiome may include for example, bacteria, archaea, protists, fungi, and viruses. Microbiome may include commensal organisms that exist within a human being without causing harm or disease. Microbiome may include organisms that are not harmful but rather harm the human when they produce toxic metabolites such as trimethylamine. Microbiome may include pathogenic organisms that cause host damage through virulence factors such as producing toxic by-products. Microbiome may include populations of microbes such as bacteria and yeasts that may inhabit the skin and mucosal surfaces in various parts of the body. Bacteria may include for example Firmicutes species, Bacteroidetes species, Proteobacteria species, Verrumicrobia species, Actinobacteria species, Fusobacteria species, Cyanobacteria species and the like. Archaea may include methanogens such as Methanobrevibacter smithies' and Methanosphaera stadtmanae. Fungi may include Candida species and Malassezia species. Viruses may include bacteriophages. Microbiome species may vary in different locations throughout the body. For example, the genitourinary system may contain a high prevalence of Lactobacillus species while the gastrointestinal tract may contain a high prevalence of Bifidobacterium species while the lung may contain a high prevalence of Streptococcus and Staphylococcus species.

With continued reference to FIG. 6, microbiome body measurement may include one or more stool test results describing presence, absence, and/or measurement of microorganisms including bacteria, archaea, fungi, protozoa, algae, viruses, parasites, worms, and the like. Stool test results may contain species such as Ackerman's muciniphila, Anaerotruncus colihominis, bacteriology, Bacteroides vulgates', Bacteroides-Prevotella, Barnesiella species, Bifidobacterium longarm, Bifidobacterium species, Butyrivbrio crossotus, Clostridium species, Collinsella aerofaciens, fecal color, fecal consistency, Coprococcus eutactus, Desulfovibrio piger, Escherichia coli, Faecalibacterium prausnitzii, Fecal occult blood, Firmicutes to Bacteroidetes ratio, Fusobacterium species, Lactobacillus species, Methanobrevibacter smithii, yeast minimum inhibitory concentration, bacteria minimum inhibitory concentration, yeast mycology, fungi mycology, Odoribacter species, Oxalobacter formigenes, parasitology, Prevotella species, Pseudoflavonifractor species, Roseburia species, Ruminococcus species, Veillonella species and the like.

With continued reference to FIG. 6, microbiome body measurement may include one or more stool tests results that identify all microorganisms living in a user's gut including bacteria, viruses, archaea, yeast, fungi, parasites, and bacteriophages. Microbiome body measurement may include DNA and RNA sequences from live microorganisms that may impact a user's health. Microbiome body measurement may include high resolution of both species and strains of all microorganisms. Microbiome body measurement may include data describing current microbe activity. Microbiome body measurement may include expression of levels of active microbial gene functions. Microbiome body measurement may include descriptions of sources of disease-causing microorganisms, such as viruses found in the gastrointestinal tract such as raspberry bushy swarf virus from consuming contaminated raspberries or Pepino mosaic virus from consuming contaminated tomatoes.

With continued reference to FIG. 6, microbiome body measurement may include one or more blood test results that identify metabolites produced by microorganisms. Metabolites may include for example, indole-3-propionic acid, indole-3-lactic acid, indole-3-acetic acid, tryptophan, serotonin, kynurenine, total indoxyl sulfate, tyrosine, xanthine, 3-methylxanthine, uric acid, and the like.

With continued reference to FIG. 6, microbiome body measurement may include one or more breath test results that identify certain strains of microorganisms that may be present in certain areas of a user's body. This may include for example, lactose intolerance breath tests, methane-based breath tests, hydrogen-based breath tests, fructose-based breath tests, Helicobacter pylori breath test, fructose intolerance breath test, bacterial overgrowth syndrome breath tests and the like.

With continued reference to FIG. 6, microbiome body measurement may include one or more urinary analysis results for certain microbial strains present in urine. This may include for example, urinalysis that examines urine specific gravity, urine cytology, urine sodium, urine culture, urinary calcium, urinary hematuria, urinary glucose levels, urinary acidity, urinary protein, urinary nitrites, bilirubin, red blood cell urinalysis, and the like.

With continued reference to FIG. 6, nutrient as used herein, includes any substance required by the human body to function. Nutrients may include carbohydrates, protein, lipids, vitamins, minerals, antioxidants, fatty acids, amino acids, and the like. Nutrients may include for example vitamins such as thiamine, riboflavin, niacin, pantothenic acid, pyridoxine, biotin, folate, cobalamin, Vitamin C, Vitamin A, Vitamin D, Vitamin E, and Vitamin K. Nutrients may include for example minerals such as sodium, chloride, potassium, calcium, phosphorous, magnesium, sulfur, iron, zinc, iodine, selenium, copper, manganese, fluoride, chromium, molybdenum, nickel, aluminum, silicon, vanadium, arsenic, and boron.

With continued reference to FIG. 6, nutrients may include extracellular nutrients that are free floating in blood and exist outside of cells. Extracellular nutrients may be located in serum. Nutrients may include intracellular nutrients which may be absorbed by cells including white blood cells and red blood cells.

With continued reference to FIG. 6, nutrient body measurement may include one or more blood test results that identify extracellular and intracellular levels of nutrients. Nutrient body measurement may include blood test results that identify serum, white blood cell, and red blood cell levels of nutrients. For example, nutrient body measurement may include serum, white blood cell, and red blood cell levels of micronutrients such as Vitamin A, Vitamin B1, Vitamin B2, Vitamin B3, Vitamin B6, Vitamin B12, Vitamin B5, Vitamin C, Vitamin D, Vitamin E, Vitamin K1, Vitamin K2, and folate.

With continued reference to FIG. 6, nutrient body measurement may include one or more blood test results that identify serum, white blood cell and red blood cell levels of nutrients such as calcium, manganese, zinc, copper, chromium, iron, magnesium, copper to zinc ratio, choline, inositol, carnitine, methylmalonic acid (MMA), sodium, potassium, asparagine, glutamine, serine, coenzyme q10, cysteine, alpha lipoic acid, glutathione, selenium, eicosapentaenoic acid (EPA), docosahexaenoic acid (DHA), docosapentaenoic acid (DPA), total omega-3, lauric acid, arachidonic acid, oleic acid, total omega 6, and omega 3 index.

With continued reference to FIG. 6, nutrient body measurement may include one or more salivary test results that identify levels of nutrients including any of the nutrients as described herein. Nutrient body measurement may include hair analysis of levels of nutrients including any of the nutrients as described herein.

With continued reference to FIG. 6, genetic as used herein, includes any inherited trait. Inherited traits may include genetic material contained with DNA including for example, nucleotides. Nucleotides include adenine (A), cytosine (C), guanine (G), and thymine (T). Genetic information may be contained within the specific sequence of an individual's nucleotides and sequence throughout a gene or DNA chain. Genetics may include how a particular genetic sequence may contribute to a tendency to develop a certain disease such as cancer or Alzheimer's disease.

With continued reference to FIG. 6, genetic body measurement may include one or more results from one or more blood tests, hair tests, skin tests, urine, amniotic fluid, buccal swabs and/or tissue test to identify a user's particular sequence of nucleotides, genes, chromosomes, and/or proteins. Genetic body measurement may include tests that example genetic changes that may lead to genetic disorders. Genetic body measurement may detect genetic changes such as deletion of genetic material or pieces of chromosomes that may cause Duchenne Muscular Dystrophy. Genetic body measurement may detect genetic changes such as insertion of genetic material into DNA or a gene such as the BRCA1 gene that is associated with an increased risk of breast and ovarian cancer due to insertion of 2 extra nucleotides. Genetic body measurement may include a genetic change such as a genetic substitution from a piece of genetic material that replaces another as seen with sickle cell anemia where one nucleotide is substituted for another. Genetic body measurement may detect a genetic change such as a duplication when extra genetic material is duplicated one or more times within a person's genome such as with Charcot-Marie Tooth disease type 1. Genetic body measurement may include a genetic change such as an amplification when there is more than a normal number of copies of a gene in a cell such as HER2 amplification in cancer cells. Genetic body measurement may include a genetic change such as a chromosomal translocation when pieces of chromosomes break off and reattach to another chromosome such as with the BCR-ABL1 gene sequence that is formed when pieces of chromosome 9 and chromosome 22 break off and switch places. Genetic body measurement may include a genetic change such as an inversion when one chromosome experiences two breaks and the middle piece is flipped or inverted before reattaching. Genetic body measurement may include a repeat such as when regions of DNA contain a sequence of nucleotides that repeat a number of times such as for example in Huntington's disease or Fragile X syndrome. Genetic body measurement may include a genetic change such as a trisomy when there are three chromosomes instead of the usual pair as seen with Down syndrome with a trisomy of chromosome 21, Edwards syndrome with a trisomy at chromosome 18 or Patau syndrome with a trisomy at chromosome 13. Genetic body measurement may include a genetic change such as monosomy such as when there is an absence of a chromosome instead of a pair, such as in Turner syndrome.

With continued reference to FIG. 6, genetic body measurement may include an analysis of COMT gene that is responsible for producing enzymes that metabolize neurotransmitters. Genetic body measurement may include an analysis of DRD2 gene that produces dopamine receptors in the brain. Genetic body measurement may include an analysis of ADRA2B gene that produces receptors for noradrenaline. Genetic body measurement may include an analysis of 5-HTTLPR gene that produces receptors for serotonin. Genetic body measurement may include an analysis of BDNF gene that produces brain derived neurotrophic factor. Genetic body measurement may include an analysis of 9p21 gene that is associated with cardiovascular disease risk. Genetic body measurement may include an analysis of APOE gene that is involved in the transportation of blood lipids such as cholesterol. Genetic body measurement may include an analysis of NOS3 gene that is involved in producing enzymes involved in regulating vasodilation and vasoconstriction of blood vessels.

With continued reference to FIG. 6, genetic body measurement may include ACE gene that is involved in producing enzymes that regulate blood pressure. Genetic body measurement may include SLCO1B1 gene that directs pharmaceutical compounds such as statins into cells. Genetic body measurement may include FUT2 gene that produces enzymes that aid in absorption of Vitamin B12 from digestive tract. Genetic body measurement may include MTHFR gene that is responsible for producing enzymes that aid in metabolism and utilization of Vitamin B9 or folate. Genetic body measurement may include SHMT1 gene that aids in production and utilization of Vitamin B9 or folate. Genetic body measurement may include MTRR gene that produces enzymes that aid in metabolism and utilization of Vitamin B12. Genetic body measurement may include MTR gene that produces enzymes that aid in metabolism and utilization of Vitamin B12. Genetic body measurement may include FTO gene that aids in feelings of satiety or fulness after eating. Genetic body measurement may include MC4R gene that aids in producing hunger cues and hunger triggers. Genetic body measurement may include APOA2 gene that directs body to produce ApoA2 thereby affecting absorption of saturated fats. Genetic body measurement may include UCP1 gene that aids in controlling metabolic rate and thermoregulation of body. Genetic body measurement may include TCF7L2 gene that regulates insulin secretion. Genetic body measurement may include AMY1 gene that aids in digestion of starchy foods. Genetic body measurement may include MCM6 gene that controls production of lactase enzyme that aids in digesting lactose found in dairy products. Genetic body measurement may include BCMO1 gene that aids in producing enzymes that aid in metabolism and activation of Vitamin A. Genetic body measurement may include SLC23A1 gene that produces and transport Vitamin C. Genetic body measurement may include CYP2R1 gene that produces enzymes involved in production and activation of Vitamin D. Genetic body measurement may include GC gene that produces and transport Vitamin D. Genetic body measurement may include CYP1A2 gene that aids in metabolism and elimination of caffeine. Genetic body measurement may include CYP17A1 gene that produces enzymes that convert progesterone into androgens such as androstenedione, androstendiol, dehydroepiandrosterone, and testosterone.

With continued reference to FIG. 6, genetic body measurement may include CYP19A1 gene that produces enzymes that convert androgens such as androstenedione and testosterone into estrogens including estradiol and estrone. Genetic body measurement may include SRD5A2 gene that aids in production of enzymes that convert testosterone into dihydrotestosterone. Genetic body measurement may include UFT2B17 gene that produces enzymes that metabolize testosterone and dihydrotestosterone. Genetic body measurement may include CYP1A1 gene that produces enzymes that metabolize estrogens into 2 hydroxy-estrogen. Genetic body measurement may include CYP1B1 gene that produces enzymes that metabolize estrogens into 4 hydroxy-estrogen. Genetic body measurement may include CYP3A4 gene that produces enzymes that metabolize estrogen into 16 hydroxy-estrogen. Genetic body measurement may include COMT gene that produces enzymes that metabolize 2 hydroxy-estrogen and 4 hydroxy-estrogen into methoxy estrogen. Genetic body measurement may include GSTT1 gene that produces enzymes that eliminate toxic by-products generated from metabolism of estrogens. Genetic body measurement may include GSTM1 gene that produces enzymes responsible for eliminating harmful by-products generated from metabolism of estrogens. Genetic body measurement may include GSTP1 gene that produces enzymes that eliminate harmful by-products generated from metabolism of estrogens. Genetic body measurement may include SOD2 gene that produces enzymes that eliminate oxidant by-products generated from metabolism of estrogens.

With continued reference to FIG. 6, metabolic, as used herein, includes any process that converts food and nutrition into energy. Metabolic may include biochemical processes that occur within the body. Metabolic body measurement may include blood tests, hair tests, skin tests, amniotic fluid, buccal swabs and/or tissue test to identify a user's metabolism. Metabolic body measurement may include blood tests that examine glucose levels, electrolytes, fluid balance, kidney function, and liver function. Metabolic body measurement may include blood tests that examine calcium levels, albumin, total protein, chloride levels, sodium levels, potassium levels, carbon dioxide levels, bicarbonate levels, blood urea nitrogen, creatinine, alkaline phosphatase, alanine amino transferase, aspartate amino transferase, bilirubin, and the like.

With continued reference to FIG. 6, metabolic body measurement may include one or more blood, saliva, hair, urine, skin, and/or buccal swabs that examine levels of hormones within the body such as 11-hydroxy-androstereone, 11-hydroxy-etiocholanolone, 11-keto-androsterone, 11-keto-etiocholanolone, 16 alpha-hydroxyestrone, 2-hydroxyestrone, 4-hydroxyestrone, 4-methoxyestrone, androstanediol, androsterone, creatinine, DHEA, estradiol, estriol, estrone, etiocholanolone, pregnanediol, pregnanestriol, specific gravity, testosterone, tetrahydrocortisol, tetrahydrocrotisone, tetrahydrodeoxycortisol, allo-tetrahydrocortisol.

With continued reference to FIG. 6, metabolic body measurement may include one or more metabolic rate test results such as breath tests that may analyze a user's resting metabolic rate or number of calories that a user's body burns each day rest. Metabolic body measurement may include one or more vital signs including blood pressure, breathing rate, pulse rate, temperature, and the like. Metabolic body measurement may include blood tests such as a lipid panel such as low density lipoprotein (LDL), high density lipoprotein (HDL), triglycerides, total cholesterol, ratios of lipid levels such as total cholesterol to HDL ratio, insulin sensitivity test, fasting glucose test, Hemoglobin A1C test, adipokines such as leptin and adiponectin, neuropeptides such as ghrelin, pro-inflammatory cytokines such as interleukin 6 or tumor necrosis factor alpha, anti-inflammatory cytokines such as interleukin 10, markers of antioxidant status such as oxidized low-density lipoprotein, uric acid, paraoxonase 1. Persons skilled in the art, upon reviewing the entirety of this disclosure, will be aware of various additional examples of physiological state data that may be used consistently with descriptions of systems and methods as provided in this disclosure.

With continued reference to FIG. 6, physiological data may be obtained from a physically extracted sample. A “physical sample” as used in this example, may include any sample obtained from a human body of a user. A physical sample may be obtained from a bodily fluid and/or tissue analysis such as a blood sample, tissue, sample, buccal swab, mucous sample, stool sample, hair sample, fingernail sample and the like. A physical sample may be obtained from a device in contact with a human body of a user such as a microchip embedded in a user's skin, a sensor in contact with a user's skin, a sensor located on a user's tooth, and the like. Physiological data may be obtained from a physically extracted sample. A physical sample may include a signal from a sensor configured to detect physiological data of a user and record physiological data as a function of the signal. A sensor may include any medical sensor and/or medical device configured to capture sensor data concerning a patient, including any scanning, radiological and/or imaging device such as without limitation x-ray equipment, computer assisted tomography (CAT) scan equipment, positron emission tomography (PET) scan equipment, any form of magnetic resonance imagery (MRI) equipment, ultrasound equipment, optical scanning equipment such as photo-plethysmographic equipment, or the like. A sensor may include any electromagnetic sensor, including without limitation electroencephalographic sensors, magnetoencephalographic sensors, electrocardiograma sensors, electromyographic sensors, or the like. A sensor may include a temperature sensor. A sensor may include any sensor that may be included in a mobile device and/or wearable device, including without limitation a motion sensor such as an inertial measurement unit (IMU), one or more accelerometers, one or more gyroscopes, one or more magnetometers, or the like. At least a wearable and/or mobile device sensor may capture step, gait, and/or other mobility data, as well as data describing activity levels and/or physical fitness. At least a wearable and/or mobile device sensor may detect heart rate or the like. A sensor may detect any hematological parameter including blood oxygen level, pulse rate, heart rate, pulse rhythm, blood sugar, and/or blood pressure. A sensor may be configured to detect internal and/or external biomarkers and/or readings. A sensor may be a part of apparatus 600 or may be a separate device in communication with apparatus 600. User data may include a profile, such as a psychological profile, generated using previous item selections by the user; profile may include, without limitation, a set of actions and/or navigational actions performed as described in further detail below, which may be combined with biological extraction data and/or other user data for processes as described in further detail below.

Physiological data and/or other data of each user may be stored, without limitation, in a user database. A user database may include any data structure for ordered storage and retrieval of data, which may be implemented as a hardware or software module. A user database may be implemented, without limitation, as a relational database, a key-value retrieval datastore such as a NOSQL database, or any other format or structure for use as a datastore that a person skilled in the art would recognize as suitable upon review of the entirety of this disclosure. A user database may include a plurality of data entries and/or records corresponding to user tests as described above. Data entries in a user database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a user database may reflect categories, cohorts, and/or populations of data consistently with this disclosure. A user database may be located in memory of computing device 104 and/or on another device in and/or in communication with apparatus 600.

With continued reference to FIG. 6, in some embodiments, apparatus may be configured to classify user data 604 to a profile cluster 612. For the purposes of this disclosure, a “profile cluster” is a category including a plurality of related phenotypes. For example, in some embodiments, a profile cluster may include a grouping of phenotypes with similar nutritional needs. In some embodiments, user data 604 may be classified to a profile cluster 612 using a phenotype classifier. Phenotype classifier may be consistent with any classifier disclosed in this disclosure. In some embodiments, phenotype classifier may be generated using a machine-learning module, such as machine-learning module 200 disclosed with respect to FIG. 2. In some embodiments, phenotype classifier may be trained using training data correlating a plurality of user data to a plurality of profile clusters. Phenotype classifier may be configured to accept user data as input and to generate a profile cluster for the user data.

With continued reference to FIG. 6, in some embodiments, apparatus 600 may be configured to classify user data 604 to profile cluster 612 as a function of a profile cluster look-up table. In some embodiments, profile cluster lookup table may relate user data to profile clusters. In some embodiments, profile cluster lookup table may relate ranges of certain types of user data to profile clusters.

Still referring to FIG. 6, apparatus 600 may assign a user one or more cohort labels as a function of one or more phenotypic clusters. As used in this disclosure, “cohort label” is an identifier assigned to a user based on a phenotypic cluster. As a non-limiting example, cohort label may further classify a user within a phenotype group. In some embodiments, cohort label may be assigned once processor 104 receives additional data. Additional data may provide more insight into a health status of a user. In some instances, cohort label may be assigned as a function of a biological extraction. As a non-limiting example, cohort label may be “diabetic” when biological extraction data indicates a high blood sugar, apparatus 600 may reference a cohort label lookup table to assign a cohort label to a user. In some embodiments, cohort label may have a biological extraction threshold. As a non-limiting example, apparatus 600 may assign a user a “anemic” when a user's iron levels fall below a threshold.

Still referring to FIG. 6, in some embodiments, apparatus 600 may be configured to receive edible data 608. “Edible data” as used in this disclosure is information relating to consumable items. In some embodiments, edible data 608 may include a meal identification. A “meal identification,” also referred to as meal ID, as used in this disclosure, is a classification of a recipe. In some embodiments, apparatus 600 may receive edible data 608 and/or a meal ID from a user. In some embodiments, a meal ID of edible data 608 may include a title and/or a description related to a meal. For example a meal ID may include a name of a dish, such as “Beef Stroganoff”. A description of a meal ID may include one or more general contents of a dish and/or a specific description of the dish. For example, a description may include data that Beef Stroganoff is an originally Russian dish of sautéed pieces of beef served in a sauce of mustard and smetana (sour cream). Still referring to FIG. 6, apparatus 600 is configured to receive recipe data containing nutrient data from the user. Recipe data may include a list of nutrients to prepare a meal. For example, and without limitation, edible data 608 may include nutrients in a beef stroganoff dish, which may include: 1 pound uncooked wide egg noodles, ¼ cup butter, divided, 2½ pounds thinly-sliced steak, fine sea salt and freshly-cracked black pepper, 4.5 small white onions, thinly sliced, 3 pound sliced mushrooms, 2 cloves garlic, minced or pressed, ½ cup dry white wine, 1½ cups beef stock, 1 tablespoon Worcestershire sauce, 3 tablespoons all-purpose flour, ½ cup of sour cream, and chopped fresh parsley.

Still referring to FIG. 6, in some embodiments, apparatus 600 may extract plurality of nutrients 616 from edible data 608 . . . “Nutrients,” as used in this disclosure, are elements of a meal. Nutrients 116 may include, but are not limited to, meats, vegetables, sauces, syrups, seafoods, fruits, dairy products, and the like. In some embodiments, apparatus 600 may utilize a language processing module to extract a plurality of nutrients 616 from edible data 608. A language processing module may include any hardware and/or software module. A language processing module may be configured to extract, from the one or more documents, one or more words, letters, characters, and the like, without limitation. One or more words may include, without limitation, strings of one or more characters, including without limitation any sequence or sequences of letters, numbers, punctuation, diacritic marks, engineering symbols, geometric dimensioning and tolerancing (GD&T) symbols, chemical symbols and formulas, spaces, whitespace, and other symbols, including any symbols usable as textual data as described above. Textual data may be parsed into tokens, which may include a simple word (sequence of letters separated by whitespace) or more generally a sequence of characters as described previously. The term “token,” as used herein, refers to any smaller, individual groupings of text from a larger source of text; tokens may be broken up by word, pair of words, sentence, or other delimitation. These tokens may in turn be parsed in various ways. Textual data may be parsed into words or sequences of words, which may be considered words as well. Textual data may be parsed into “n-grams”, where all sequences of n consecutive characters are considered. Any or all possible sequences of tokens or words may be stored as “chains”, for example for use as a Markov chain or Hidden Markov Model.

Still referring to FIG. 6, a language processing module may operate to produce a language processing model. A language processing model may include a program automatically generated by computing device and/or a language processing module to produce associations between one or more words extracted from at least a document and detect associations, including without limitation mathematical associations, between such words. Associations between language elements, where language elements include for purposes herein extracted words, relationships of such categories to other such term may include, without limitation, mathematical associations, including without limitation statistical correlations between any language element and any other language element and/or language elements. Statistical correlations and/or mathematical associations may include probabilistic formulas or relationships indicating, for instance, a likelihood that a given extracted word indicates a given category of semantic meaning. As a further example, statistical correlations and/or mathematical associations may include probabilistic formulas or relationships indicating a positive and/or negative association between at least an extracted word and/or a given semantic meaning; positive or negative indication may include an indication that a given document is or is not indicating a category semantic meaning. Whether a phrase, sentence, word, or other textual element in a document or corpus of documents constitutes a positive or negative indicator may be determined, in an embodiment, by mathematical associations between detected words, comparisons to phrases and/or words indicating positive and/or negative indicators that are stored in memory at computing device, or the like. In some embodiments, language processing module may be configured to identify tokens corresponding to nutrients or categories of nutrients within a corpus of text. Language processing module may be trained using training data containing a plurality of tokens representing nutrients and/or categories of nutrients. In some embodiments, the language processing module training data may include input texts correlated to tokens representing nutrients and/or categories of nutrients extracted from those texts.

Still referring to FIG. 6, a language processing module and/or diagnostic engine may generate the language processing model by any suitable method, including without limitation a natural language processing classification algorithm; a language processing model may include a natural language process classification model that enumerates and/or derives statistical relationships between input terms and output terms. Algorithm to generate language processing model may include a stochastic gradient descent algorithm, which may include a method that iteratively optimizes an objective function, such as an objective function representing a statistical estimation of relationships between terms, including relationships between input terms and output terms, in the form of a sum of relationships to be estimated. In an alternative or additional approach, sequential tokens may be modeled as chains, serving as the observations in a Hidden Markov Model (HMM). HMMs as used herein are statistical models with inference algorithms that that may be applied to the models. In such models, a hidden state to be estimated may include an association between an extracted words, phrases, and/or other semantic units. There may be a finite number of categories to which an extracted word may pertain; an HMM inference algorithm, such as the forward-backward algorithm or the Viterbi algorithm, may be used to estimate the most likely discrete state given a word or sequence of words. Language processing module may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

Continuing to refer to FIG. 6, generating a language processing model may include generating a vector space, which may be a collection of vectors, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each vector in an n-dimensional vector space may be represented by an n-tuple of numerical values. Each unique extracted word and/or language element as described above may be represented by a vector of the vector space. In an embodiment, each unique extracted and/or other language element may be represented by a dimension of vector space; as a non-limiting example, each element of a vector may include a number representing an enumeration of co-occurrences of the word and/or language element represented by the vector with another word and/or language element. Vectors may be normalized, scaled according to relative frequencies of appearance and/or file sizes. In an embodiment associating language elements to one another as described above may include computing a degree of vector similarity between a vector representing each language element and a vector representing another language element; vector similarity may be measured according to any norm for proximity and/or similarity of two vectors, including without limitation cosine similarity, which measures the similarity of two vectors by evaluating the cosine of the angle between the vectors, which can be computed using a dot product of the two vectors divided by the lengths of the two vectors. Degree of similarity may include any other geometric measure of distance between vectors.

Still referring to FIG. 6, a language processing module may use a corpus of documents to generate associations between language elements in a language processing module, and diagnostic engine may then use such associations to analyze words extracted from one or more documents and determine that the one or more documents indicate significance of a category. In an embodiment, language module and/or apparatus 600 may perform this analysis using a selected set of significant documents, such as documents identified by one or more experts as representing good information; experts may identify or enter such documents via graphical user interface, or may communicate identities of significant documents according to any other suitable method of electronic communication, or by providing such identity to other persons who may enter such identifications into apparatus 600. Documents may include recipes, nutrient lists, nutritional facts, or the like. Documents may include words and alphanumeric character strings associated with edible data. Documents may include words and alphanumeric character strings associated with nutrient data. Documents may be entered into a computing device by being uploaded by an expert or other persons using, without limitation, file transfer protocol (FTP) or other suitable methods for transmission and/or upload of documents; alternatively or additionally, where a document is identified by a citation, a uniform resource identifier (URI), uniform resource locator (URL) or other datum permitting unambiguous identification of the document, diagnostic engine may automatically obtain the document using such an identifier, for instance by submitting a request to a database or compendium of documents such as JSTOR as provided by Ithaka Harbors, Inc. of New York.

Still referring to FIG. 6, in some embodiments, apparatus 600 may utilize optical character recognition to identify and/or extract plurality of nutrients 616 from edible data 608. Optical character recognition (OCR) is disclosed further with reference to FIG. 1.

Still referring to FIG. 6, in some embodiments, edible data 608 may be provided by a user, such as, but not limited to, a chef, line cook, an individual, and the like. Edible data 608 may be received and/or stored by a graphical user interface or a user database as described further below. Alternatively or additionally, apparatus 600 may retrieve edible data 608 from an online repository or other suitable source for retrieving information regarding meal preparation. In non-limiting illustrative examples, edible data 608 may contain sequentially ordered tasks that may be sequentially ordered based upon a chronological order, tasks ordered by resource optimization, and the like. Edible data 608 may contain elements, steps, instructions, or the like that refer to preparing one or more meals, by one or more personnel, using one of more stations, appliances, utensils, and the like. In non-limiting illustrative examples, apparatus 600 may retrieve a plurality of edible data 608 by retrieving a series of steps corresponding to a meal ID, for instance and without limitation, recipe steps using available nutrients 616 for cooking a beef stew. In further non-limiting illustrative examples, the steps to a beef stew may be associated with a chronological sequential ordering of personnel tasks, nutrient retrieval, kitchen space use, and may differ based upon time constraints, including and/or avoiding certain nutrients 616, equipment, and the like.

Still referring to FIG. 6, in some embodiments, apparatus 600 may be configured to generate a web search. A “web search” as used in this disclosure is a query for information through the Internet. Generating a web search may include generating a web crawler function. A web search may be configured to search for one or more keywords, key phrases, and the like. A keyword may be used by a query to filter potential results from a query. As a non-limiting example, a keyword may include “Gluten”. A query may be configured to generate one or more key words and/or phrases as a function of edible data 608. A query may give a weight to one or more semantic elements of edible data 608. “Weights”, as used herein, may be multipliers or other scalar numbers reflecting a relative importance of a particular attribute or value. A weight may include, but is not limited to, a numerical value corresponding to an importance of an element. In some embodiments, a weighted value may be referred to in terms of a whole number, such as 1, 100, and the like. As a non-limiting example, a weighted value of 0.2 may indicate that the weighted value makes up 20% of the total value. As a non-limiting example, edible data 608 may include the words “gluten free”. A query may give a weight of 0.8 to the word “gluten”, and a weight of 0.2 to the word “free”. A query may map a plurality of semantic elements of query results having similar elements to the word “gluten” with differing elements than the word “free” due to the lower weight value paired to the word “gluten”. In some embodiments, a query may pair one or more weighted values to one or more semantic elements of edible data 608. Weighted values may be tuned through a machine-learning model, such as any machine learning model as described throughout this disclosure without limitation. In some embodiments, a query may generate weighted values based on prior queries. In some embodiments, a query may be configured to filter out one or more “stop words” that may not convey meaning, such as “of,” “a,” “an,” “the,” or the like.

Still referring to FIG. 6, in some embodiments, apparatus 600 may generate an index classifier. In an embodiment, an index classifier may include a classifier. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. An index classifier may include a classifier configured to input semantic elements and output web search indices. A “web search index,” as defined in this disclosure is a data structure that stores uniform resource locators (URLs) of web pages together with one or more associated data that may be used to retrieve URLs by querying the web search index; associated data may include keywords identified in pages associated with URLs by programs such as web crawlers and/or “spiders.” A web search index may include any data structure for ordered storage and retrieval of data, which may be implemented as a hardware or software module. A web search index may be implemented, without limitation, as a relational database, a key-value retrieval datastore such as a NOSQL database, or any other format or structure for use as a datastore that a person skilled in the art would recognize as suitable upon review of the entirety of this disclosure. Data entries in a web search index may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a web search index may reflect categories, cohorts, and/or populations of data consistently with this disclosure. In an embodiment, a web search query at a search engine may be submitted as a query to a web search index, which may retrieve a list of URLs responsive to the query. In some embodiments, apparatus 600 may be configured to generate a query based on a freshness and/or age of a query result. A freshness may include an accuracy of a query result. An age may include a metric of how outdated a query result may be. In some embodiments, apparatus 600 may generate a web crawler configured to search the Internet for edible data 608, such as, but not limited to, nutrients 616, preparation steps, category of food, allergen data, and the like. As a non-limiting example, a query may include a web crawler configured to search and/or index information of words and/or phrases having a similarity to edible data 608.

Still referring to FIG. 6, in some embodiments, edible data 608 may include nutrient data. “Nutrient data,” as used in this disclosure, is information pertaining to the nutritional value of one or more nutrients 616. In some embodiments, nutrient data may include nutritional values related to nutrients 616 in a meal. For example, nutritional values may include the value of vitamin, caloric, protein, fat, cholesterol, sugar, carbohydrate, sodium, and the like in the meal. For example, in beef stroganoff, the nutritional values may be calories 235, total fat 11 g, saturated fat 6 g, cholesterol 50 mg, sodium 1,044 mg, potassium 336 mg, total carbohydrate 22 g, dietary fiber 1.4 g, sugar 4 g, protein 12 g, vitamin c. In some embodiments, nutritional values may include a daily value of nutrients 616 in a dish. “Daily value (DV),” as used in this disclosure, is the recommended amount of nutrients 616 a person should consume and not to exceed each day. The % DV may be how much a nutrient in a single serving of an individual dish or dietary supplement contributes to a daily diet. For example, if the DV for a certain nutrient is 300 micrograms (mcg) and a dish or supplement has 30 mcg in one serving, the % DV for that nutrient in a serving of the product may be 10%. In some embodiments, apparatus 600 may receive recipe data from a user database. User database may contain recipe data received from a plurality of different users categorized to a common meal ID. For example, user database may contain a recipe data table containing a plurality of different recipes and nutritional values common for a beef stroganoff dish.

Still referring to FIG. 6, in some embodiments, apparatus 600 may be configured to classify plurality of nutrients 616 to impact factors 620. An “impact factor,” as used in this disclosure, is a metric of influence one or more nutrients 616 has on an individual's biological system. A “biological system” as used in this disclosure is a process and/or group of processes that occur in an individual's physiology. Impact factors 620 may include, without limitation, concentration of nutrients 616, quantity of nutrients 616, calories of nutrients 616, allergens associated with one or more nutrients 616, carbohydrate and/or other macronutrient quantities, ratios, and the like. In some embodiments, impact factors 620 may be based on essential macronutrients and micronutrients. “Micronutrients,” as used herein, are nutrients that a person needs in small doses. For example, micronutrients may include vitamins and minerals. “Macronutrients,” as used herein, are nutrients that a person needs in larger amounts. For example, macronutrients may include water, protein, carbohydrates, and fats. In some embodiments, impact factors 620 may be based on nutrients 616 essential for boosting the immune system, helping prevent or delay certain cancers, such as prostate cancer, strengthening teeth and bones, aiding in calcium absorption, maintaining healthy skin, helping the body metabolize proteins and carbs, supporting healthy blood, burning fat, building muscle, maintaining weight, losing water weight, aiding brain and nervous system functioning, aiding in blood clotting, helping to carry oxygen and/or the like. A user may select, through GUI 628, what impact factors 620 may be based on. In some embodiments, a user may select a plurality of impact factors 620. In some embodiments, receive impart factor data in the form of documents, medical papers, research papers, and the like through an impact factors 620 database. “Impact factor database,” as used in this disclosure, is a data structure containing information related to a plurality of impact factors 620. An impact factor database may be populated by apparatus 600 utilizing a web crawler. An impact factor database may be populated by expert submission. An “expert,” as used herein, is a person who has a comprehensive and authoritative knowledge of or skill in a particular area. For example, a doctor may submit a paper on how fish oil aids in preventing cancer. An expert submission may include a single expert submission and/or a plurality of submissions from an expert; plurality of submissions may be received from a plurality of experts as described in U.S. patent application Ser. No. 16/397,814, filed, Apr. 29, 2019, and titled “METHODS AND SYSTEMS FOR CLASSIFICATION USING EXPERT DATA”, of which is incorporated by reference herein in its entirety.

Still referring to FIG. 6, in some embodiments, apparatus 600 may determine one or more impact factors 620 for one or more phenotypes. A “phenotype,” as used in this disclosure, is a composite observable characteristic or trait of an individual. A phenotype may include a user's biochemical and physiological properties, behavior, and products of behavior. Behavioral phenotypes may include cognitive, personality, and behavior patterns. This may include effects on cellular and physiological phenotypic traits that may occur due to external or environmental factors. For example, DNA methylation and histone modification may alter phenotypic expression of genes without altering underlying DNA sequence. Phenotype may include a congenital disorder, anomaly, and the like, such as hearing defects, trisomy 18 (Edward's syndrome), trisomy 21 (down syndrome), trisomy 13 (Patau syndrome), cleft palate, spina bifida, phenylketonuria, glutamate carboxypeptidase II mutation, pyloric stenosis, congenital hip dislocation, anencephaly, hypoplasia, Meckel's diverticulum, and the like. Phenotype may include a genotype-environment interaction (GxE). Phenotype may include any diagnosis (current disorder) and/or prognosis (predicted difficulty, future diagnosis, outcome, and the like) associated with a congenital factor. Phenotype may include identifiers associated with disorders, conditions, symptoms, and the like, which may correspond with categorization. Phenotype may include a predictive classification, where a subject may be considered reasonably healthy at birth, does not harbor congenital factor(s) indicative of obvious current congenital disorder but may include data that indicates a phenotype with which they may be most closely categorized to, and/or an imminent categorization. A phenotype may be stored and/or retrieved from a user database.

Still referring to FIG. 6, in some embodiments, apparatus 600 may classify plurality of nutrients 616 to impact factors 620 utilizing a nutrient classifier. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A nutrient classifier may be configured to output at least a datum that labels or otherwise identifies a set of data that are clustered together, found to be close under a distance metric as described below, or the like. In some embodiments, a nutrient classifier may receive meal ID and recipe data as an input and output a plurality of matched nutrient data elements to an importance factor. For example, a nutrient classifier may match nutrients 616 in a dish that contain a nutritional value that are essential for boosting the immune system. In some embodiments, a nutrient classifier may receive meal ID and recipe data as an input and output a plurality of matched nutrient data elements to a plurality of impact factors. For example, a nutrient classifier may match nutrients 616 in a dish that contain a nutritional value that are essential for boosting the immune system, building muscle, maintaining healthy skin, and the like. Training data for a nutrient classifier may include data from impact factor data. For example, classification based on muscle building may include training data containing documents and expert submission exemplifying nutrients 616 that may correlate to muscle building. In some embodiments, training data may include, a plurality of recipe data received from a plurality of user from user database.

Still referring to FIG. 6, in some embodiments, apparatus 600 may receive edible data 608 and/or a meal ID from a user database. A “user database,” as used in this disclosure is a data structure containing information uploaded by a user. User database may contain information from a plurality of different users categorized to a common meal ID. For example, user database may contain a meal ID table containing a plurality of different titles common for a beef stroganoff dish and a plurality of different meal descriptions associated to the dish. User database and all other databases in this disclosure may be implemented, without limitation, as a relational user database, a key-value retrieval user database such as a NOSQL user database, or any other format or structure for use as a user database that a person skilled in the art would recognize as suitable upon review of the entirety of this disclosure. User database may alternatively or additionally be implemented using a distributed data storage protocol and/or data structure, such as a distributed hash table or the like. User database may include a plurality of data entries and/or records as described above. Data entries in a user database may be flagged with or linked to one or more additional elements of information, which may be reflected in data entry cells and/or in linked tables such as tables related by one or more indices in a relational user database. Persons skilled in the art, upon reviewing the entirety of this disclosure, will be aware of various ways in which data entries in a user database may store, retrieve, organize, and/or reflect data and/or records as used herein, as well as categories and/or populations of data consistently with this disclosure. A user database may include one or more elements of edible data 608 and/or user data. In some embodiments, a user database may be populated through user input and/or one or more web searches.

Still referring to FIG. 6, in some embodiments, a nutrient classifier may be further configured to generate nutrient score 624. A “nutrient score” as used in this disclosure is a value given to a nutrient. A nutrient classifier may score plurality of nutrients 616. A nutrient classifier may score plurality of nutrients 616 across a plurality of phenotypes to determine an impact factor of impact factors 620. In some instances, nutrient classifier may be trained using nutrient classifier training data. In some embodiments, nutrient classifier training data may include historical nutrient data correlated to categories of nutrients. In some embodiments, nutrient classifier training data may contain categories of nutrients correlated to scores for those categories of nutrients. In some embodiments, a lookup table correlating categories of nutrients to nutrient scores 624 may be used to determine a nutrient score 624 once nutrient classifier determines a category of nutrient. In some embodiments, a nutrient classifier may be configured to score a plurality of nutrients 616 of plurality of nutrients 616. Nutrient scores 624 may be based off, without limitation, relative impact of one or more nutrients on one or more phenotypes. For instance, and without limitation, a score of 3 out of 10 may be assigned to a filet mignon for a phenotype of vegan. In some embodiments, a score may be based off a nutrition target range. In some embodiments, nutrient score 624 may be generated by using an objective function as described in further detail below. It should be noted, the nutrient score 624 may be generated using an objective function that is optimized using impact factors as constraints. In some instances, objective function may be optimized using profile clusters as constraints.

Still referring to FIG. 6, apparatus 600 may be configured to receive and/or determine a nutrition target range of one or more users, profile clusters 612, and the like. A “nutrition target range,” as used in this disclosure, is a value or range of values of quantities of nutrients 616. Apparatus 600 may utilize a nutrient target machine learning model to determine a nutrient target range of one or more individuals. A nutrient target machine learning model may be trained with training data correlating user data to nutrition target ranges. In some instances, training data may correlate a biological extraction and/or phenotypes to target ranges. In some instances, training data may correlate a user profile to target ranges. Training data may be received through user input, external computing devices, and/or previous iterations of processing. In some instances, training data may be retrieved from a database storing user data correlated to target ranges. As a non-limiting example, training data may be stored in a training data lookup table (LUT). As used in this disclosure, a “lookup table” is an array of data that maps input values to output values. A lookup table may be used to replace a runtime computation with an array indexing operation. In another non limiting example, a training data look up table may be able to relate user data to target ranges. In some embodiments, a nutrient classifier and/or apparatus 600 may utilize a nutrient target machine learning model. A nutrient target machine learning model may be configured to receive user data and/or edible data 608 and output one or more nutrient target ranges of one or more individuals and/or groups of individuals.

Apparatus 600 may utilize a nutrient score machine learning model to determine a nutrient score of at least a nutrient. A nutrient score machine learning model may be trained with training data correlating phenotype data to nutrient scores. Training data may be received through user input, external computing devices, and/or previous iterations of processing. In some instances, training data may be retrieved from a database storing user data correlated to target ranges. As a non-limiting example, training data may be stored in a training data lookup table (LUT).

Still referring to FIG. 6, computing device may be configured to generate a nutrition supplement as a function of the nutrition range target. A “nutrition supplement,” as used in this disclosure, is a modification of a recipe. Nutritional supplements may include, without limitation, different sets of nutrients 616, such as spices, meats, seasonings, vitamin powders, and the like. In some embodiments, a nutrient classifier may be configured to receive training data correlating recipe data and/or user data to one or more nutritional supplements. Training data may be received through user, external computing devices, and/or previous iterations of processing. A nutrient classifier may input edible data 608 and/or user data and output one or more nutritional supplements, suggested by computing device that offer nutritional values aligned to nutrition range target.

Still referring to FIG. 6, in some embodiments, apparatus 600 may be configured to generate nutrient chain. A “nutrient chain” as used in this disclosure is a set of edible items. Edible items may include, without limitation, seasonings, spices, vitamin powders, meats, seafood, fruits, vegetables, dairy products, and the like. In some embodiments, apparatus 600 may compare impact factors 620 with plurality of nutrients 616 to generate nutrient chain. In some embodiments, apparatus 600 may be configured to compare any data as described throughout this disclosure using an objective function. For instance, apparatus 600 may generate an objective function. An “objective function” as used in this disclosure is a process of minimizing or maximizing one or more values based on a set of constraints. In some embodiments, an objective function of apparatus 600 may include an optimization criterion. An optimization criterion may include any description of a desired value or range of values for one or more impact factors; desired value or range of values may include a maximal or minimal value, a range between maximal or minimal values, or an instruction to maximize or minimize an impact factor. As a non-limiting example, an optimization criterion may specify that an impact factor should be within a 1% difference of an optimization criterion. An optimization criterion may alternatively request that an impact factor be greater than a certain value. An optimization criterion may specify one or more tolerances for differences in macronutrients of one or more nutrients 616 in a recipe. An optimization criterion may specify one or more desired impact factor criteria for a nutrient chain. In an embodiment, an optimization criterion may assign weights to different impact factors or values associated with impact factors. One or more weights may be expressions of value to a user of a particular outcome, impact factor value, or other facet of a nutrient chain. Optimization criteria may be combined in weighted or unweighted combinations into a function reflecting an overall outcome desired by a user; function may be a nutrient chain function to be minimized and/or maximized. A function may be defined by reference to impact factor criteria constraints and/or weighted aggregation thereof as provided by apparatus 600; for instance, an impact factor function combining optimization criteria may seek to minimize or maximize a function of nutrient chain generation.

Still referring to FIG. 6, generation of an objective function may include generation of a function to score and weight factors to achieve a process score for each feasible pairing. In some embodiments, pairings may be scored in a matrix for optimization, where columns represent nutrients 616 and rows represent impact factors potentially paired therewith; each cell of such a matrix may represent a score of a pairing of the corresponding nutrient to the corresponding impact factor. In some embodiments, assigning a predicted process that optimizes the objective function includes performing a greedy algorithm process. A “greedy algorithm” is defined as an algorithm that selects locally optimal choices, which may or may not generate a globally optimal solution. For instance, apparatus 600 may select pairings so that scores associated therewith are the best score for each impact factor and/or for each nutrient. In such an example, optimization may determine the combination of nutrients 616 such that each impact factor pairing includes the highest score possible.

Still referring to FIG. 6, an objective function may be formulated as a linear objective function. Apparatus 600 may solve an objective function using a linear program such as without limitation a mixed-integer program. A “linear program,” as used in this disclosure, is a program that optimizes a linear objective function, given at least a constraint. For instance, and without limitation, objective function may seek to maximize a total score Σ_{rϵR} Σ_{sϵs} cᵣₛxᵣₛ, where R is a set of all nutrients 616 r, S is a set of all impact factors s, cᵣₛ is a score of a pairing of a given nutrient with a given impact factor, and xᵣₛ is 1 if an nutrient r is paired with an impact factor s, and 0 otherwise. Continuing the example, constraints may specify that each nutrient is assigned to only one impact factor, and each impact factor is assigned only one nutrient. Impact factors may include nutrients 616 as described above. Sets of nutrients 616 may be optimized for a maximum score combination of all generated nutrients 616. In various embodiments, apparatus 600 may determine a combination of nutrients 616 that maximizes a total score subject to a constraint that all nutrients 616 are paired to exactly one impact factor. Not all impact factors may receive a nutrient pairing since each impact factor may only produce one nutrient pairing. In some embodiments, an objective function may be formulated as a mixed integer optimization function. A “mixed integer optimization” as used in this disclosure is a program in which some or all of the variables are restricted to be integers. A mathematical solver may be implemented to solve for the set of feasible pairings that maximizes the sum of scores across all pairings; mathematical solver may be implemented on apparatus 600, another device, and/or may be implemented on third-party solver.

With continued reference to FIG. 6, optimizing an objective function may include minimizing a loss function, where a “loss function” is an expression an output of which an optimization algorithm minimizes to generate an optimal result. As a non-limiting example, apparatus 600 may assign variables relating to a set of parameters, which may correspond to score nutrients 616 as described above, calculate an output of mathematical expression using the variables, and select a pairing that produces an output having the lowest size, according to a given definition of “size,” of the set of outputs representing each of a plurality of nutrients 616 and/or impact factors; size may, for instance, included absolute value, numerical size, or the like. Selection of different loss functions may result in identification of different potential pairings as generating minimal outputs. Objectives represented in an objective function and/or loss function may include minimization of impact factors. Objectives may include minimization of preparation time of a recipe. Objectives may include minimization of costs of a recipe. Objectives may include maximization of compatibility across a wide range of individuals.

Still referring to FIG. 6, in some embodiments, apparatus 600 may receive a meal ID through a graphical user interface (GUI) 628. A “graphical user interface” as used in this disclosure is an interface including a set of one or more pictorial and/or graphical icons corresponding to one or more computer actions. GUI 628 may be configured to receive user input, as described above. GUI 628 may include one or more event handlers. In some embodiments, GUI 628 may be configured to display outputs. In some instances, GUI 628 may display a score of nutrient 616. An “event handler” as used in this disclosure is a callback routine that operates asynchronously once an event takes place. Event handlers may include, without limitation, one or more programs to perform one or more actions based on user input, such as generating pop-up windows, submitting forms, changing background colors of a webpage, and the like. Event handlers may be programmed for specific user input, such as, but not limited to, mouse clicks, mouse hovering, touchscreen input, keystrokes, and the like. For instance and without limitation, an event handler may be programmed to generate a pop-up window if a user double clicks on a specific icon. User input may include manipulation of computer icons, such as, but not limited to, clicking, selecting, dragging and dropping, scrolling, and the like. In some embodiments, user input may include an entry of characters and/or symbols in a user input field. A “user input field” as used in this disclosure is a portion of a graphical user interface configured to receive data from an individual. A user input field may include, but is not limited to, text boxes, search fields, filtering fields, and the like. In some embodiments, user input may include touch input. Touch input may include, but is not limited to, single taps, double taps, triple taps, long presses, swiping gestures, and the like. One of ordinary skill in the art will appreciate the various ways a user may interact with GUI 628. In some embodiments, GUI 628 may be consistent with graphical user interfaces as described in U.S. patent application Ser. No. 17/062,740, filed Oct. 5, 2020, and titled “METHODS AND SYSTEMS FOR ARRANGING AND DISPLAYING GUIDED RECOMMENDATIONS VIA A USER INTERFACE BASED ON BIOLOGICAL EXTRACTION”, of which is incorporated by reference herein in its entirety.

Continuing in reference to FIG. 6, apparatus 600 may retrieve a plurality of nutrient chain, wherein retrieving a nutrient chain may include retrieving, for each meal of the plurality of meals, a nutrient chain identifying a plurality of sequentially ordered tasks for preparation of the meal. A nutrient chain may be provided by a user, such as a restaurant, cook, or the like, and nutrient chains may be stored and/or retrieved by apparatus 600 from a meal database, for instance from a nutrient chain database. Alternatively or additionally, apparatus 600 may retrieve nutrient chain from an online repository or other suitable source for retrieving information regarding meal preparation. In non-limiting illustrative examples, a nutrient chain may contain sequentially ordered tasks that may be sequentially ordered based upon a chronological order, tasks ordered by resource optimization, task ordered by customer priority, and the like. A nutrient chain may contain elements, steps, instructions, or the like that refer to preparing one or more meals, by one or more personnel, using one of more stations, appliances, utensils, and the like. Apparatus 600 may store and/or retrieve nutrient chain, or an element of an existing nutrient chain to form a new nutrient chain, from a meal database, online repository, blog, culinary website, or any other suitable source, as described above. In non-limiting illustrative examples, apparatus 600 may retrieve a plurality of nutrient chain by retrieving a series of steps corresponding to an identification of a meal of edible data 608, for instance and without limitation, recipe steps using available nutrients 616 for cooking a beef stew. In further non-limiting illustrative examples, the steps to a beef stew may be associated with a chronological sequential ordering of personnel tasks, nutrient retrieval, kitchen space use, and may differ based upon time constraints, including and/or avoiding certain nutrients 616, equipment, and the like.

Continuing in reference to FIG. 6, apparatus 600 may retrieve a plurality of nutrient chain, wherein retrieving may include identifying, for each nutrient chain, a resource list identifying a plurality of resources, wherein each resource is associated with a task of the plurality of sequentially ordered tasks. A “resource list,” as described in this disclosure refers to a tabulation, list, or the like, of nutrient identities, amounts, and expirations; kitchen stations, equipment, appliances, utensils, dishware, personnel, operating hours, tables, customers; delivery couriers including restaurant employees and secondary couriers via application services, ‘gig’ economy services, and the like; delivery vehicles, including cars, trucks, bikes, and the like, and any other suitable resource relating the preparation of a meal, delivery of a meal, and/or meal orders. Apparatus 600 may determine a resource and tabulate, list, group, or otherwise categorize a plurality of resources by retrieving a resource from a database, as described above. Alternatively or additionally, a resource of a resource list may be stored and/or retrieved from a database by a machine-learning process, such as a first machine-learning model, as a resource may correspond to a plurality of meals, nutrients 616, nutrient chains, or the like.

Still referring to FIG. 6, in some embodiments, apparatus 600 may retrieve and/or generate a plurality of nutrient chain. Nutrient chain may differ at branching points that correspond to different pathways, series of elements, steps, or the like in preparing a meal. In non-limiting illustrations a branch point may represent places where deviations in tasks in a nutrient chain may differ for instance, omitting or including a step to eliminate or add a new nutrient, for instance removing onions from the beef stew upon customer request, or customizing meal by adding chives. In further non-limiting illustrative examples, a branch point may represent a place in an nutrient chain where concurrently performed steps are added, subtracted, combined, or split into new nutrient chain. For instance and without limitation, a branch point may include a beginning preparation of a beef stock for a meal, a first kitchen personnel may include next any series of vegetables, beginning with any of the four, before moving to a next task in the nutrient chain. In such an example, several nutrient chain modifications may be introduced at the branch point, for instance and without limitation, a second kitchen personnel may be added to assist in the nutrient preparation steps to decrease time of meal preparation, or a fifth nutrient may be added upon request to customize a meal further, resource permitting. Nutrient chain may be listed in a sequentially ordered manner and mapped to the anticipated timescale for preparing a meal; timescale may be altered by applying different resource lists to different steps in nutrient chain and/or modifying nutrient chain by adding/subtracting branch points, removing/adding tasks, and the like. For instance and without limitation, a plurality of nutrient chain may be mapped to a 12-hr time scale for preparing a beef stew, wherein a negative time value represents “time out” from a meal being finished, and a positive time value represents “time post preparation,” including for example delivery time, customer retrieval time, and the like. Nutrient chain may be optimized, combined, and or otherwise modified as described in further detail below to decrease average time of preparation.

Referring still to FIG. 6, apparatus 600 may generate a plurality of candidate nutrient chain combinations, wherein each nutrient chain combination may include a first nutrient chain of the plurality of nutrient chains and a second nutrient chain of the plurality of nutrient chains, and a first task of the first nutrient chain and a second task of the second nutrient chain are concurrently performed using a resource associated with each of the first task and the second task. A first task of a first nutrient chain and a second task of a second nutrient chain may be placed in a sequentially ordered sequence and/or performed concurrently relative to each other depending on constraints on task ordering and/or combination. For instance, a first task of a first nutrient chain may be to prepare a first meal at a station and a second task of a second nutrient chain may be to prepare a second meal, wherein the first meal introduces an allergen to be excluded from the second meal; this would introduce a constraint that would limit the sequential ordering in this manner. In such an example, the sequential order of the two different nutrient chains would need to be changed based on avoiding said allergen. In further non-limiting illustrative examples, a first task of a first nutrient chain may be to prepare 1 cup of a first nutrient and a second task of a second nutrient chain may be to prepare 1 cup of that same nutrient, a first task of a first nutrient chain and a second task of a second nutrient chain may be combined concurrently to improve efficiency, wherein a single person may prepare 2 cups at once. Determining if any constraint exists may include determining if a constraint would limit a first task of a first nutrient chain being ordered followed by a second task in a second nutrient chain in either a sequential and/or concurrent ordering. If either nutrient chain ordering is determined to be allowed based upon constraints, then ordering of a plurality of nutrient chains may be added to a feasible list for further feasibility quantifier analysis, as described in further detail below. In non-limiting illustrative examples, a plurality of nutrient chain may be concurrently listed, for instance combined into a plurality of candidate nutrient chain combinations determined by sequentially listing certain tasks and concurrently performing other tasks within a potential combination of nutrient chains, wherein concurrently performed tasks overlap at least for a moment in time, personnel, station, equipment, or overlap in any resource, as described above. In further non-limiting illustrative examples, concurrently performed tasks of two nutrient chains may involve, for instance and without limitation, a combination of resource at once such as preheating an oven of a first nutrient chain, washing utensils to remove allergens of a second nutrient chain, and chopping vegetables of a third nutrient chain. In such a non-limiting example, a first nutrient chain may correspond to preparing two distinct meals, such as a second meal and third meal, each of which may require heating in an oven at the same temperature, or at an average temperature suitable for both meals while using a single oven, wherein the average temperature is an optimized temperature calculated by a machine-learning model and/or objective function to batch cooking steps together, as described in further detail below; a second nutrient chain may correspond to removing allergens from a first meal that can be done while preparing a second meal and a third meal, but must be completed prior to finishing the preheating stage; a third nutrient chain may correspond to chopping vegetables that may correspond to an nutrient preparation task that overlaps with a plurality of meals.

Continuing in reference to FIG. 6, nutrient chain and candidate nutrient chain combinations may include signifiers, numerical values, alphanumerical codes, and the like that contain elements of data regarding identifiers related to certain combinations of nutrient chains elements, resource amounts, time amounts, constraints, and/or any other identifiable parameters that may be used in determining feasibility of an nutrient chain, or plurality of nutrient chain combinations, as described in further detail below. A machine-learning model may, for instance, retrieve nutrient chain from a meal database and determine feasibility of said nutrient chain by identification by a signifier, as described above.

Continuing in reference to FIG. 6, apparatus 600 may identify a plurality of constraints as a function of identifications of meals, which may include at least a resource constraint and at least a timing constraint. A “constraint,” as used herein refers to a barrier, limitation, consideration, or any other constraint pertaining to resource utilization during optimizing the combination of a plurality of nutrient chains that may arise during meal preparation and/or delivery as a function of performing a plurality of nutrient chains combinations, wherein the constraint may alter the time and/or resources available to preparing a meal or performing a task, may alter the concurrent and/or sequential ordering of tasks in a plurality of nutrient chains, and/or may alter the feasibility of combining a plurality of nutrient chains. Constraints may be identified by an optimization process during optimization of nutrient chain combinations, as described in further detail below. A constraint may, for instance and without limitation, only appear during a particular optimized listing of a plurality of nutrient chain elements, wherein a second listing of the same elements in a different ordering may not show the same constraint. In non-limiting illustrative examples, a constraint may be a resource constraint, wherein dedicating an individual to a series of tasks for preparing a meal would then place a constraint on preparing a second meal with said individual, or performing a second combination of nutrient chains; likewise a constraint may be a time constraint wherein the maximal time allotted for selecting nutrient chains for an individual or set of individuals working in tandem in preparing a meal may be dictated by when a customer places an order, whether a customer is dine-in or take-out, delivery method for the meal, and/or type of meal and nutrients 616 used. Constraints may refer to customer preferences, for instance and without limitation, such as the presence of allergies, food intolerances, hypersensitivities, or other dietary constraints, philosophical, religious, and/or moral considerations to nutrients 616 and/or meal preparation, and the like; constraints may refer to seasonality of nutrients 616, nutrient amounts, nutrient substitutions, and/or other material and immaterial constraints to nutrient availability and use. Such information may be stored and/or retrieved by apparatus 600 from a database, for instance, via orders input by a restaurant wait staff, logged by a web-based application, mobile application, or other meal ordering service, application, device, of the like. Meal orders may be provided in a non-electronic format and nutrient chain retrieved after a user prompts apparatus 600 for nutrient chain associated with an order, which may contain constraint information. Constraint information may be stored and/or retrieved alongside nutrient chain information by use of an alphanumeric code, numerical value, or any other method of signifying the presence, amount, and/or nature of a constraint related to a task, nutrient chain, and/or combinations of nutrient chain.

Continuing in reference to FIG. 6, apparatus 600 may be configured to generate plurality of candidate nutrient chain combinations by receiving feasibility training data. Feasibility training data may include a plurality of entries correlating task combinations with feasibility quantifiers. A “feasibility quantifier,” as used in this disclosure is a score, metric, function, vector, matrix, numerical value, or the like, which describes a qualitative and/or quantitative mathematical proportion, propensity, or any relationship correlating the likelihood, possibility, and/or probability of completing a task, given a set of constraints and the a task's relationship in time to and ordering to other tasks, within a particular timeframe, wherein a timeframe may be determined by an identification of a meal, meal preparation time, expected delivery time, resource list, nutrient chain, and the like. Apparatus 600 may identify feasible combinations based on various constraints, wherein apparatus 600 may find and/or set values for those constraints or add a new constraint in the form of a “feasibility quantifier”. In non-limiting illustrative examples, a feasibility quantifier may include scores relating the probability of feasibility for completing a series of tasks, wherein each task has an associated probability in relating preparation of a beef stew related to preparing the beef stew for a customer order within, for instance, a 15-minute time frame, 30-minute time frame, 1-hour time frame, etc. In further non-limiting illustrative examples, nutrient chain steps that would require more than a 15-minute time frame would garner scores indicating lower levels of feasibility, such as for instance placing beef in a marinade, chopping vegetables, and cooking a beef stock, and thus may result in candidate nutrient chain steps that would sequentially order meal preparation of such steps for a suitable amount of time prior to the 15-minute time frame. Additionally, in non-limiting illustrative examples, nutrient chain steps that could be completed within the 15-minute time frame of ordering may include combining the nutrients 616, and plating the meal, which would garner higher feasibility scores resulting in nutrient chain elements that may be combined in such a way that allows an individual to complete the entire nutrient chain combination to fulfill orders within 15 minutes of the customer placing the order. Feasibility quantifiers may be stored and/or retrieved from a database. Alternatively or additionally, determining the feasibility of an nutrient chain may include resource constraint information, as described in further detail below, wherein the feasibility of an individual completing a first candidate nutrient chain combination depends upon if that same individual is dedicated to a second candidate nutrient chain combination, and if the suitable kitchenware, utensils, appliances, workstations, and the like, are in use and/or if preparation of the next meal may result in an biological and/or philosophical conflict for a customer, for instance an allergy to peanuts, a lactose-free meal after cooking with milk, Kosher preparation, or a vegan meal. Feasibility quantifiers may incorporate information, for instance, if there would be enough time to prepare a second meal after a first meal, if a second meal would demand decontamination of a common space to avoid antigen cross-contamination. In such a non-limiting illustrative example, a feasibility quantifier may rank a candidate nutrient chain combination in such a way that gave a more favorable score to preparing a second meal first, followed by a first meal, as described in further detail below.

With continued reference to FIG. 6, apparatus 600 may be configured to generate a recipe machine-learning model. A “recipe machine-learning model,” as used in this disclosure, is a mathematical representation of a relationship between inputs and outputs, as generated using any machine-learning process and/or machine-learning algorithm including without limitation any process as described herein, and stored in memory; an input is submitted to a machine-learning model once created, which generates an output based on the relationship that was derived. Generating recipe machine-learning model may include calculating one or more supervised machine-learning algorithms including active learning, classification, regression, analytical learning, artificial neural network, backpropagation, boosting, Bayesian statistics, case-based learning, genetic programming, Kernel estimators, naïve Bayes classifiers, maximum entropy classifier, conditional random field, K-nearest neighbor algorithm, support vector machine, random forest, ordinal classification, data pre-processing, statistical relational learning, and the like. Generating recipe machine-learning model may include calculating one or more unsupervised machine-learning algorithms, including a clustering algorithm such as hierarchical clustering, k-means clustering, mixture models, density based spatial clustering of algorithms with noise (DBSCAN), ordering points to identify the clustering structure (OPTICS), anomaly detection such as local outlier factor, neural networks such as autoencoders, deep belief nets, Hebbian learning, generative adversarial networks, self-organizing map, and the like. Generating recipe machine-learning model may include calculating a semi-supervised machine-learning algorithm such as reinforcement learning, self-learning, feature learning, sparse dictionary learning, anomaly detection, robot learning, association rules and the like. Recipe machine-learning model is trained by apparatus 600 using training data, including any of the training data as described herein. Training data may be obtained from records of previous iterations of generating recipe machine-learning model, user inputs and/or questionnaire responses, expert inputs, and the like. Recipe machine-learning model may be implemented as any machine-learning process, including for instance, and without limitation, as described in U.S. Nonprovisional application Ser. No. 16/375,303, filed on Apr. 4, 2019, and entitled “SYSTEMS AND METHODS FOR GENERATING ALIMENTARY INSTRUCTION SETS BASED ON VIBRANT CONSTITUTIONAL GUIDANCE,” the entirety of which is incorporated herein by reference. Recipe machine-learning model is trained using training data to select recommended refreshments favored by a user selection. In an embodiment, user selection contained within a selection database may be utilized as training data to customize and train recipe machine-learning model individually for each user. For instance and without limitation, user selection that indicate a user prefers to eat foods that contain protein choices that contain either chicken, tofu or salmon and the user dislikes protein choices that contain beef or pork may be utilized as training data to generate recommended refreshments such as chicken picada, tofu and green bean stir fry, and miso glazed salmon, and to not generate recommended refreshments such as a ground beef stir fry or a pork burger. In another embodiment, recipe machine-learning model may output a plurality of recommended refreshments as a function of the health condition of the user. For instance and without limitation, a user may have a gluten allergy. In this example, recipe machine-learning model may output recommended refreshment suitable for the user where the recommended refreshment are gluten-free.

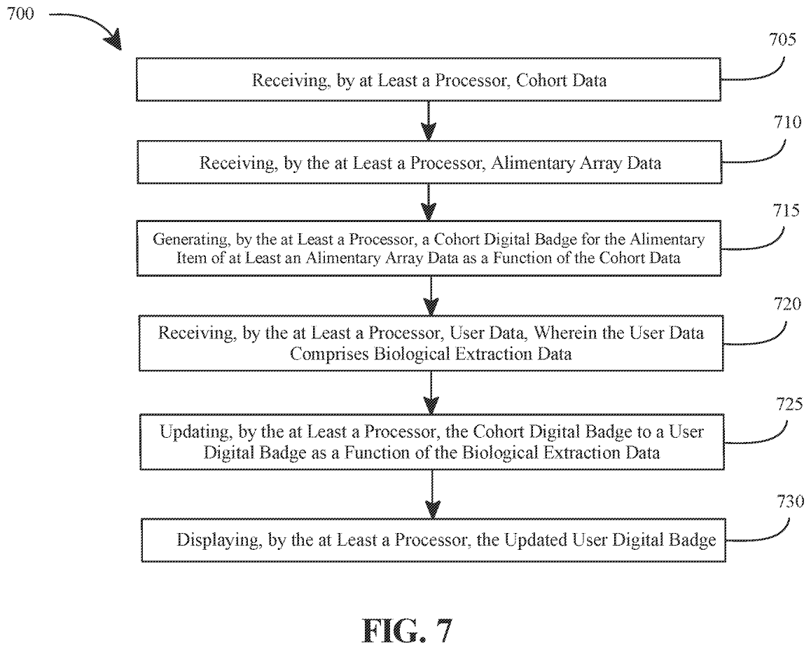

Referring now to FIG. 7, a flow diagram of an exemplary method 700 for generating a customized badge is illustrated. Method 700 includes step 705 of receiving, by at least a processor, cohort data. This may be implemented, without limitation, as described above with reference to FIGS. 1-6.

With continued reference to FIG. 7, method 700 includes a step 710 of receiving, by the at least a processor, alimentary array data. This may be implemented, without limitation, as described above with reference to FIGS. 1-6.

With continued reference to FIG. 7, method 700 includes a step 715 of generating, by the at least a processor, a cohort digital badge for the alimentary item of at least alimentary array data as a function of the cohort data. This may be implemented, without limitation, as described above with reference to FIGS. 1-6.

With continued reference to FIG. 7, method 700 includes a step 720 of receiving, by the at least a processor, user data, wherein the user data comprises biological extraction data. This may be implemented, without limitation, as described above with reference to FIGS. 1-6.

With continued reference to FIG. 7, method 700 includes a step 725 of updating, by the at least a processor, the cohort digital badge to a user digital badge as a function of the biological extraction data. This may be implemented, without limitation, as described above with reference to FIGS. 1-6.

With continued reference to FIG. 7, method 700 includes a step 730 of displaying, by the at least a processor, the updated user digital badge. This may be implemented, without limitation, as described above with reference to FIGS. 1-6.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

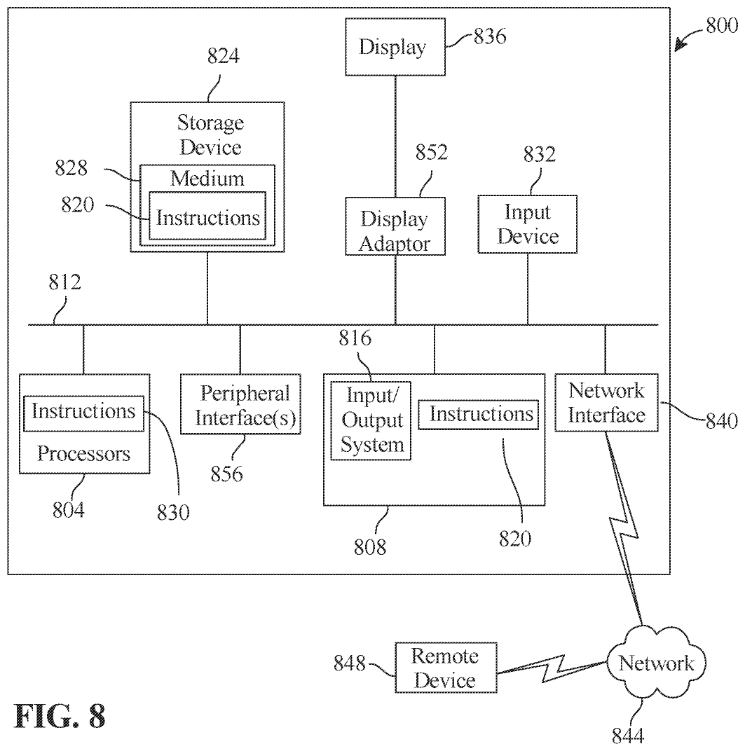

FIG. 8 shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components thereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a user of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a user selection device for selecting one or more graphical representations in a graphical interface as described above.

A user may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A system for generating a customized badge, wherein the system comprises:
at least a processor; and
a memory communicatively connected to the at least a processor, the memory containing instructions configuring the processor to:
receive cohort data;
receive alimentary array data, wherein the alimentary array data comprises food quality standards;
generate a cohort digital badge for an alimentary item of the alimentary array data as a function of the cohort data;
receive user data associated with a user, wherein the user data comprises biological extraction data;
update the cohort digital badge to a user digital badge as a function of the biological extraction data;
display the updated user digital badge, wherein the updated user digital badge incorporates color-coded indicators that display adherence of the user to specific dietary guidelines.

2. The system of claim 1, wherein displaying the updated user digital badge further comprises:
generating a digital menu as a function of the alimentary array; and
displaying the updated user digital badge on the digital menu at a display corresponding to the alimentary item.

3. The system of claim 1, wherein displaying the updated user digital badge further comprises:
scanning a physical menu;
identifying a listing on the physical menu corresponding to the alimentary item;
generating a combined display of the physical menu and the updated user digital badge,
wherein the combined display displays the updated user digital badge at the listing; and
displaying the combined display.

4. The system of claim 1, wherein the system is further configured to integrate environmental data into the user digital badge, wherein integrating the environmental data into the user digital badge comprises:
training an environmental score machine-learning model using environmental score training data, wherein the environmental score training data comprises a plurality of environmental data correlated to environmental scores;
receiving environmental data for the alimentary item;
generating an environmental score using the environmental data and the environmental score machine-learning model; and
incorporating the environmental score into the cohort digital badge.

5. The system of claim 1, wherein updating the cohort digital badge to a user digital badge comprises generating the user digital badge, which comprises selecting a preferred alimentary item as a function of user score data, wherein the preferred alimentary item is selected for having higher user score data compared to another alimentary item.

6. The system of claim 1, wherein generating a customized badge further comprises updating the cohort digital badge to the user digital badge as a function of user feedback.

7. The system of claim 1, wherein the memory comprises instructions further configuring at least a processor to:
adjust the user digital badge, wherein adjusting the user digital badge comprises:
receiving a digital badge machine-learning model;
training the digital badge machine-learning model using user-specific training data; and
generating the updated user digital badge using the trained digital badge machine-learning model.

8. The system of claim 1, the system is configured to use a web crawler to:
collect validation data as a function of the at least alimentary array data; and
generating the cohort badge as a function of the validation data.

9. The system of claim 1, wherein the system is further configured to determine cohort nutrient data using a phenotype, wherein generating the cohort digital badge comprises generating the cohort digital badge as a function of the cohort nutrient data.

10. The system of claim 1, wherein displaying the user digital badge comprises:
generating an alimentary display data structure comprising the at least alimentary array data and an alimentary array event handler, wherein:
the alimentary display data structure is configured to cause a display device to display the alimentary array and the user digital badge; and
the alimentary array event handler is configured to detect a user interaction on an item of the at least an alimentary array and, as a function of the detection, display a user score associated with the item; and
transmit the alimentary display data structure to the display device.

11. A method for generating a customized badge, wherein the method comprises:
receiving, by at least a processor, cohort data;
receiving, by the at least a processor, alimentary array data, wherein the alimentary array data comprises food quality standards;
generating, by the at least a processor, a cohort digital badge for an alimentary item of the alimentary array data as a function of the cohort data;
receiving, by the at least a processor, user data associated with a user, wherein the user data comprises biological extraction data;
updating, by the at least a processor, the cohort digital badge to a user digital badge as a function of the biological extraction data;
displaying, by the at least a processor, the updated user digital badge, wherein the updated digital badge incorporates color-coded indicators that display adherence of the user to specific dietary guidelines.

12. The method of claim 11, wherein displaying the updated user digital badge further comprises:
generating a digital menu as a function of the alimentary array; and
displaying the updated user digital badge on the digital menu at a display corresponding to the alimentary item.

13. The method of claim 11, wherein displaying the updated user digital badge further comprises:
scanning a physical menu;
identifying a listing on the physical menu corresponding to the alimentary item;
generating a combined display of the physical menu and the updated user digital badge, wherein the combined display displays the updated user digital badge at the listing; and
displaying the combined display.

14. The method of claim 11, further comprising integrating, by the at least a processor, environmental data into the user digital badge, wherein integrating the environmental data into the user digital badge comprises:
training an environmental score machine-learning model using environmental score training data, wherein the environmental score training data comprises a plurality of environmental data correlated to environmental scores;
receiving environmental data for the alimentary item;
generating an environmental score using the environmental data and the environmental score machine-learning model; and
incorporating the environmental score into the cohort digital badge.

15. The method of claim 11, wherein updating the cohort digital badge to a user digital badge generating the user digital badge, which comprises selecting a preferred alimentary item as a function of user score data, wherein the preferred alimentary item is selected for having higher user score data compared to another alimentary item.

16. The method of claim 11, wherein generating a customized badge further comprises updating the cohort digital badge to the user digital badge as a function of user feedback.

17. The method of claim 11, further comprising adjusting, by the at least a processor, the user digital badge, wherein adjusting the user digital badge comprises:
receiving a digital badge machine-learning model;
training the digital badge machine-learning model using user-specific training data; and
generating the updated user digital badge using the trained digital badge machine-learning model.

18. The method of claim 11, further comprising using, by the at least a processor, a web crawler to:
collect validation data as a function of the at least alimentary array data; and
generating the cohort badge as a function of the validation data.

19. The method of claim 11, further comprising determining, by the at least a processor, cohort nutrient data using a phenotype, wherein generating the cohort digital badge comprises generating the cohort digital badge as a function of the cohort nutrient data.

20. The method of claim 11, wherein displaying the user digital badge comprises:
generating an alimentary display data structure comprising the at least alimentary array data and an alimentary array event handler, wherein:
the alimentary display data structure is configured to cause a display device to display the alimentary array and the user digital badge; and
the alimentary array event handler is configured to detect a user interaction on an item of the at least an alimentary array and, as a function of the detection, display a user score associated with the item; and
transmit the alimentary display data structure to the display device.
